(19) United States
(12) Patent Application Publication
(10) Pub. No.: US 2026/0364257 A1
(43) Pub. Date: Nov. 19, 2026

(54) CASSETTE WITH TELESCOPIC NEEDLE SHIELD FOR AN AUTO INJECTOR

(71) Applicant: Phillips-Medisize A/S, Struer (DK)

(72) Inventors: Jan Olesen, Holstebro (DK); Bjorn Knud Andersen, Struer (DK)

(21) Appl. No.: 19/135,397

(22) PCT Filed: Dec. 11, 2023
(86) PCT No.: PCT/EP2023/085170

§ 371 Date: Jun. 4, 2025

(30) Foreign Application Priority Data
Dec. 12, 2022 (EP) 22212842.3
Dec. 12, 2022 (EP) 22212843.1

(51) Int. Cl. A61M 5/20; A61M 5/24; A61M 5/32; A61M 5/34

(52) U.S. Cl. CPC A61M 5/20; A61M 5/24; A61M 5/3202; A61M 5/344; A61M 2205/197

(57) ABSTRACT

The invention relates to a cassette with a telescopic needle shield, wherein the cassette is adapted for being received in an auto injector.

### BACKGROUND

The invention relates to a cassette with a telescopic needle shield, wherein the cassette is adapted for being received in an auto injector.

Auto injectors for the delivery of medicament to a patient comes in many varieties depending on the type of medicament, which is to be delivered to the patient. When the auto injector is a re-usable injector part into which a disposable cassette is inserted, there are some main requirements, which need to be fulfilled for each of the two parts. For the cassette, there need to be mechanical stability, but at the same time, a minimum of material use in the disposable cassette is often also desired in order to keep production costs and the environmental impact low. Patent application WO 2021/069106 A1 discloses an example of a system comprising a reusable auto injector and a disposable cassette, where the disposable cassette is absent of major electronic parts and spring systems.

### SUMMARY

Different type of medicaments sets forward different requirements in terms of storage conditions. Some types of medicament may deteriorate or be damaged in other ways if coming into contact with metal for a longer period of time. This sets forward a number of challenges in terms of storage of a cassette with a syringe containing this type of medicament, as connection between an injection needle and a syringe compartment containing the medicament may have unwanted consequences. Other cassette system may easily allow for the syringe to include a needle already connected to and having fluid connection with the medicament inside the syringe compartment.

The auto injector, which is used in combination with the cassette, is normally customized for the dimensions of the different functional parts in the cassette with a purpose of reducing the number of parts in the cassette to a minimum in order to obtain the largest amount of stability in the system. Still, reusable auto injectors carry a significant level of flexibility, because they can easily be programmed by software to be optimized for delivery of different medicaments. The programmable variations may include the injection delivery duration, the maximum plunger force and similar. However, the optimization of the auto injector sets forward limits on the design of the cassette.

Disclosed herein in a first aspect is therefore a cassette for use in an auto injector for administering a medicament, wherein the cassette extends in a longitudinal direction (L) from a proximal end to a distal end. The cassette comprises a syringe holder configured for receiving a syringe containing the medicament. The syringe may be comprising a seal at a proximal end of the syringe, the seal being configured for being pierced by a needle to obtain a fluid communication between the medicament in the syringe and the needle. The syringe holder comprises a needle assembly securing part at a proximal end of the syringe holder, wherein the needle assembly securing part is configured for securing a needle assembly comprising the needle to the syringe holder after mounting of the cassette in an auto injector.

The cassette further comprises:
a first needle safe cover;
a second needle safe cover, wherein the first needle safe cover is displaceable relative to the second needle safe cover along the longitudinal direction; and
one or more release arms, wherein the one or more release arms are movable between at least two positions relative to the syringe holder, the at least two positions including:
   an initial position (PraIN) where the first needle safe cover and the second needle safe cover are separated by at the most a first separation distance; and
   a release position (PraR) where the first needle safe cover and the second needle safe cover are allowed to move relatively into a second separation distance being larger than the first separation.

By the cassette according to the first aspect, it is possible to use an auto injector originally designed and optimized for receiving a cassette system with a syringe having a pre-mounted needle for delivery medicament from a cassette system with a syringe requiring the user to connect an injection needle to the syringe compartment at the users end. The flexibility of the auto injector, which is typically limited to the cassette being provided in a certain physical format is therefore mitigated. This reduces the production cost of the reusable auto injector, as the modification to the cassette provides for that the same reusable injector can support medicament delivery processes using different cassette types. For example, if there is an absence of a pre-mounted needle, the cassette according to the first aspect ensures that the needle safe covers can allow both for mounting the needle as well as a coverage of the needle after medicament delivery using the same auto injector as used for medicament delivery from a cassette with a pre-mounted needle. Introducing any changes to the auto injector is by the above described cassette avoided. Making changes to the software control processes of an electronic reusable auto injector is normally fairly easy because they can be made without influencing mechanical structures of such a system. However, often an application of alternative cassettes requires mechanical alterations to the auto injector, which by the above cassette is avoided. Thus, capturing and handling specific requirements and needs of medicament delivery from specific cassettes leaving the reusable auto injector unmodified is possible by the cassette as described above. This lover the cost of purchasing the complex and durable reusable auto injector.

The telescopic functionality of the needle safe cover system further ensures that the needle safe cover can be extended as to allow for both securing an injection needle to the cassette and for covering the needle after end medicament delivery.

The one or more release arms may be part of the second needle safe cover or the first needle safe cover. The one or more release arms may be substituted by release fingers, a release protrusion or any other release means, which control the relative movement of the first needle safe cover and the second needle safe cover between the initial position and the release position. The second separation distance may be an extended position. The relative movement between the two needle safe cover parts may be seen as a telescopic movement.

The proximal movement of the first needle safe cover may allow the two needle safe covers to extend telescopically. The distance between the first needle safe cover and the second needle safe cover thereby increases.

Disclosed herein in a second aspect is a cassette for use in an auto injector for administering a medicament, wherein the cassette extends in a longitudinal direction (L) from a proximal end to a distal end. The cassette comprises:
a needle safe cover;
an index ring rotatable relative to the needle safe cover between a plurality of rotational positions including at least:
   a first rotational position (R1) in which the needle safe cover is prevented from moving distally; and
   a second rotational position (R2) in which the needle safe cover is longitudinally moveable;
a syringe holder configured for receiving a syringe containing the medicament, the syringe comprising a seal at a proximal end of the syringe, the seal being configured for being pierced by a needle to obtain a fluid communication between the medicament in the syringe and the needle.

The syringe holder according to the second aspect may comprise a needle assembly securing part at a proximal end of the syringe holder, wherein the needle assembly securing part is configured for securing a needle assembly comprising the needle to the syringe holder after mounting of the cassette in an auto injector.

The cassette according to the second aspect may further comprise one or more index ring release tabs movable between at least two positions relative to the index ring, the at least two positions including:
an initial position (PraIN) where the index ring is prevented from rotating from the first rotational position (R1) to the second rotational position (R2); and
a release position (PraR) where the index ring is rotatable from the first rotational position (R1) to the second rotational position (R2);
   wherein movement of one or more release members of the auto injector into a first release member position inside the cassette, when the cassette is secured in the auto injector, moves the one or more index ring release tabs from the initial position (PraIN) to the release position (PraR).

By the cassette according to the second aspect, it is possible to use an auto injector originally designed and optimized for receiving a cassette system with a syringe having a pre-mounted needle for delivery medicament from a cassette system with a syringe requiring the user to connect an injection needle to the syringe compartment at the users end. The flexibility of the auto injector, which is typically limited to the cassette being provided in a certain physical format is therefore mitigated. This reduces the production cost of the reusable auto injector, as the modification to the cassette provides for that the same reusable injector can support medicament delivery processes using different cassette types. For example, if there is an absence of a pre-mounted needle, the cassette according to the second aspect ensures that the needle safe covers can allow both for mounting the needle as well as a coverage of the needle after medicament delivery using the same auto injector as used for medicament delivery from a cassette with a pre-mounted needle. Introducing any changes to the auto injector is by the above described cassette avoided. Making changes to the software control processes of an electronic reusable auto injector is normally fairly easy because they can be made without influencing mechanical structures of such a system. However, often an application of alternative cassettes requires mechanical alterations to the auto injector, which by the above cassette is avoided. Thus, capturing and handling specific requirements and needs of medicament delivery from specific cassettes leaving the reusable auto injector unmodified is possible by the cassette as described above. This lover the cost of purchasing the complex and durable reusable auto injector.

Disclosed herein in a third aspect is further a cassette for use in an auto injector for administering a medicament, wherein the cassette extends in a longitudinal direction (L) from a proximal end to a distal end. The cassette comprises a syringe holder configured for receiving a syringe containing the medicament. The syringe may be comprising a seal at a proximal end of the syringe, the seal being configured for being pierced by a needle to obtain a fluid communication between the needle and the medicament in the syringe. The syringe holder comprises a needle assembly securing part at a proximal end of the syringe holder, wherein the needle assembly securing part is configured for securing a needle assembly comprising the needle to the syringe holder after mounting of the cassette in an auto injector. The cassette further comprises a needle mounting blocking system, wherein a proximal end of the needle mounting blocking system is configured for moving between a plurality of longitudinal positions relative to the syringe holder. The plurality of longitudinal positions at least comprises:
a locked position (PnsL) preventing piercing of the seal by the needle; and
a needle assembly connecting position (PnsC) where piercing of the seal by the needle is allowed.

The movement of the proximal end of the needle mounting blocking system is configured to be controlled by the auto injector after securing the cassette in the auto injector.

By “controlled by the auto injector after securing the cassette in the auto injector” is meant that the movement of the needle mounting blocking system is prevented until the auto injector changes part of the configuration of the cassette thereby allowing the needle mounting blocking system to move distally.

By the cassette according to the third aspect, is therefore obtained that any premature contact between an injection needle and the medicament inside the syringe compartment is prevented. It is simply not possible for a user to secure a needle to the syringe and thereby bring the injection needle into fluid contact with the medicament before the user has secured the cassette in the auto injector. This prevents situations, where the user would connect a needle assembly to a cassette with the purpose of being prepared for a fast injection at a later point in time. By the cassette according to the third aspect, it is also possible to use an auto injector originally designed and optimized for receiving a cassette system with a syringe having a pre-mounted needle for delivery medicament from a cassette system with a syringe requiring the user to connect an injection needle to the syringe compartment at the users end. The flexibility of the auto injector, which is typically limited to the cassette being provided in a certain physical format is therefore mitigated. This reduces the production cost of the reusable auto injector, as the modification to the cassette provides for that the same reusable injector can support medicament delivery processes using different cassette types. For example, if there is an absence of a pre-mounted needle, the cassette according to the first aspect and/or second aspect ensures that the needle safe covers can allow both for mounting the needle as well as a coverage of the needle after medicament delivery using the same auto injector as used for medicament delivery from a cassette with a pre-mounted needle. Introducing any changes to the auto injector is by the above described cassette avoided. Making changes to the software control processes of an electronic reusable auto injector is normally fairly easy because they can be made without influencing mechanical structures of such a system. However, often an application of alternative cassettes requires mechanical alterations to the auto injector, which by the above cassette is avoided. Thus, capturing and handling specific requirements and needs of medicament delivery from specific cassettes leaving the reusable auto injector unmodified is possible by the cassette as described above. This lover the cost of purchasing the complex and durable reusable auto injector.

The syringe configured for being received in the cassette according to the first aspect or the second aspect or the third aspect may comprise a syringe compartment containing the medicament. The compartment may be a one-chamber compartment.

Alternatively, the syringe compartment may be a two-chamber compartment comprising a first sub-compartment containing a first medicament component and a second sub-compartment containing a second medicament component, wherein a second stopper is separating the two sub-compartments. In one or more examples, the syringe compartment comprises:
a first sub-compartment containing a first medicament component;
a second sub-compartment containing a second medicament component;
a by-pass region positioned between the first sub-compartment and the second sub-compartment;
a first stopper positioned distally in the first sub-compartment before use; and
a second stopper positioned between the first sub-compartment and the second sub-compartment before use;
   wherein when moving in the first stopper in the proximal direction, the first medicament component is moved proximally thereby moving the second stopper proximally allowing for the first medicament component to enter the second sub-compartment through the by-pass region, whereby the first medicament component and the second medicament component are mixed.

The medicament in the first medicament component and the second sub-compartment may be mixed prior to medicament delivery by proximal movement of second stopper.

Alternatively, the syringe two-chamber compartment may be designed to hold two liquid substances which are injected subsequently, e.g. through the same injection needle.

In one or more examples, the needle assembly securing part secures the needle assembly to the proximal end of the syringe holder by a threaded connection, or a press fit connection, or a snap fit connection, or a bayonet connection, or a combination of these. Alternative connection options may also be imagined. the defined options are only examples.

The needle assembly connecting position (PnsC) is normally distal of the locked position (PnsL).

In one or more examples, the needle assembly is pre-mounted at the proximal end of the needle mounting blocking system prior to use, and the fluid connection between the syringe and the needle is obtained by distal movement of the needle assembly and the needle mounting blocking system relative to the syringe, possibly combined with a rotational movement of the needle assembly relative to the syringe. Alternatively, or in combination, the needle assembly may be pre-mounted at the first needle safe cover prior to use, and the fluid connection between the syringe and the needle is obtained by distal movement of the needle assembly, the second needle safe cover and the first needle safe cover relative to the syringe, possibly combined with a rotational movement of the needle assembly relative to the syringe. By having the needle assembly pre-mounted at the proximal end of the needle mounting blocking system and/or the first needle safe cover is meant that the needle assembly may be resting and supported at the proximal end of the needle mounting blocking system and/or the first needle safe cover. Thereby, the user may have a pre-prepared system, where he/she does not need to keep track of where the needle assembly is. Instead, the needle assembly and the cassette may be stored together as one unit, where the needle penetration of the seal is still prevented until the cassette and needle assembly system is secured in the auto injector.

In one or more examples, the proximal end of the needle mounting blocking system is configured as a needle safe cover covering the needle after medicament delivery. The needle mounting blocking system thereby has a double functionality.

In one or more examples, the needle mounting blocking system comprises:
a first needle safe cover;
a second needle safe cover, wherein the first needle safe cover is displaceable relative to the second needle safe cover along the longitudinal direction; and
one or more release arms, wherein the one or more release arms are movable between at least two positions relative to the syringe holder, the at least two positions including:
   an initial position (PraIN) where the first needle safe cover and the second needle safe cover are separated by at the most a first separation distance; and
   a release position (PraR) where the first needle safe cover and the second needle safe cover are allowed to move relatively into a second separation distance being larger than the first separation.

The one or more release arms may be part of the second needle safe cover or the first needle safe cover. The one or more release arms may be substituted by release fingers, a release protrusion or any other release means, which control the relative movement of the first needle safe cover and the second needle safe cover between the initial position and the release position. The second separation distance may be an extended position. The relative movement between the two needle safe cover parts may be seen as a telescopic movement.

In one or more examples, the proximal end of the needle mounting blocking system is comprised in the second needle safe cover. Constructions, where the first needle safe cover comprises the proximal end of the needle mounting blocking system may also be imagined. Which of the two covers parts that prevents mounting of the needle assembly is normally not critical as long as one of the parts includes the function.

In one or more examples, after securing the cassette in the auto injector, the second needle safe cover is configured for being longitudinally unlocked, whereby the second needle safe cover is moveable in the distal direction relative to the syringe holder thereby moving the proximal end of the needle mounting blocking system and/or the second needle safe cover into the needle assembly connecting position (PnsC) allowing for securing of the needle assembly to the syringe holder. The unlocking of the needle mounting blocking system and/or the second needle safe cover allows for mounting of the needle assembly to the cassette.

In one or more examples, after securing the cassette in the auto injector, the first needle safe cover and the one or more cover springs are configured for being longitudinally unlocked, whereby the first needle safe cover and the one or more cover springs are moveable in the distal direction relative to the syringe holder.

In one or more examples, the cassette, e.g. the needle mounting blocking system, further comprises one or more cover springs biased between the first needle safe cover and the second needle safe cover. The cover springs may be substituted by a spring system inside the auto injector acting on the first needle safe cover relative to the second needle safe cover after mounting of the cassette in the auto injector. Thus, after relative release of two needle safe cover part, movement from the initial position (PraIN) to the release position (PraR) may be assisted by the one or more cover springs in the cassette or by a spring system inside the auto injector.

In one or more examples, when the proximal end of the needle mounting blocking system is in the locked position (PnsL) preventing piercing of the seal by the needle, the second needle safe cover is longitudinally locked relative to the syringe holder. The second needle safe cover may thereby prevent piercing of the seal by the needle. In constructions, where the first needle safe cover comprises the proximal end of the needle mounting blocking system, the first needle safe cover will normally be the cover part, which is longitudinally locked relative to the syringe holder in the locked position (PnsL).

In one or more examples, the plurality of longitudinal positions, which the proximal end of the needle mounting blocking system is moveable between, further comprises a medicament delivery position (PnsD) where the needle is exposed; and a final locked position (PnsFL) where further longitudinal movement of the needle mounting blocking system is prevented, wherein the final locked position (PnsFL) is proximal of the medicament delivery position (PnsD). The extended locked position of the needle mounting blocking system may allow the needle mounting blocking system to also be utilized as a needle shield system, where the injection needle may be blocked after medicament delivery.

In one or more examples, the plurality of longitudinal positions, which the proximal end of the needle mounting blocking system is moveable between, further comprises a ready for use position (PnsR) where the proximal end of the needle mounting blocking system covers the needle, wherein the needle mounting blocking system is in the ready for use position (PnsR) prior to moving to the medicament delivery position (PnsD). This protects the user and/or a medical practitioner delivering the medicament to a patient from accidental contact with the needle prior to initiating medicament delivery.

In one or more examples, the cassette according to the first aspect further comprises an index ring rotatable relative to first needle safe cover between a plurality of rotational positions including at least:
a first rotational position (R1) in which the second needle safe cover is prevented from moving distally; and
a second rotational position (R2) in which the second needle safe cover is longitudinally moveable;
   wherein movement of one or more release members in the auto injector when the cassette is secured in the auto injector rotates the index ring. The index ring may provide for an easy unlocking of the cassette parts, where the index ring rotation is controlled by longitudinal movement of the one or more release members of the auto injector into/out of the cassette.

Likewise, in one or more examples, the cassette according to the third aspect further comprises an index ring rotatable relative to the needle mounting blocking system between a plurality of rotational positions including at least:
a first rotational position (R1) in which the needle mounting blocking system is prevented from moving distally; and
a second rotational position (R2) in which the needle mounting blocking system is moveable between the locked position (PnsL) and the needle assembly connecting position (PnsC);
   wherein rotation of the index ring is controlled by movement of one or more release members in the auto injector when the cassette is secured in the auto injector. The index ring may provide for an easy unlocking of the needle mounting blocking system, where the index ring rotation is controlled by longitudinal movement of the one or more release members of the auto injector into/out of the cassette.

The index ring according to all aspects of the cassette may comprise a track extending from a first corner to a second corner of a surface area of the index ring.

In one or more examples, the index ring according to any of the aspects is configured for rotating from the first rotational position (R1) to the second rotational position (R2) when the cassette is inserted in the auto injector and the one or more release members comprised in the auto injector travels proximally inside the track. Introducing inclining surfaces in the track allows for an easy rotation of the index ring. Normally, the index ring is prevented from moving in the longitudinal direction relative to the syringe holder.

In one or more examples, the track extends from the first corner in an angled direction towards the second corner, wherein the angled direction is between 20 and 70 degrees, such as between 30 and 60 degrees, such as between 35 and 55 degrees, such as between 40 and 50 degrees, such as approximately 45 degrees relative to the longitudinal direction of the cassette. The track may be one track having the same angled direction angle along the entire track. Alternatively, the angled direction need not extend over the entire track portion or have the same angle over the entire track portion. Instead, it may have separate track parts with different angled directions.

In one or more examples, the track comprises a first track section and a second track section, wherein:
the first track section extends from the first corner in a first angled direction towards a first middle point;
the second track section extends from the first middle point to a second middle point along the longitudinal direction of the cassette in a second angled direction.

The first angled direction may be between 20 and 70 degrees, such as between 30 and 60 degrees, such as between 35 and 55 degrees, such as between 40 and 50 degrees, such as approximately 45 degrees relative to the longitudinal direction of the cassette.

The second angled direction may be between −20 and 20 degrees, such as between −10 and 10 degrees, such as approximately 0 degrees relative to the longitudinal direction of the cassette.

This creates two track portions, where proximal movement of the one or more release members into and along the first track portion will rotate the index ring from the first rotational position R1 to the second rotational position R2. When the one or more release members travels distally, the index ring will rotate back to the first rotational position. When the one or more release members travels in and along the second track portion, the index ring is not rotated further if the second angled direction is approximately 0 degrees.

In one or more examples, the track further comprises a third track section, wherein the third track section extends from the second middle point in a third angled direction towards the second corner. The third angled direction may be between 20 and 70 degrees, such as between 30 and 60 degrees, such as between 35 and 55 degrees, such as between 40 and 50 degrees, such as approximately 45 degrees relative to the longitudinal direction of the cassette. Including a third track section allows for a further rotation of the index ring from the second rotation position to a third rotation position. This provides for an opportunity to include further releasing and/or securing of cassette parts to the index ring.

In one or more examples, the cassette further comprises one or more index ring release tabs movable relative to the index ring, wherein movement of one or more release members of the auto injector into a first release member position inside the cassette, when the cassette is secured in the auto injector, moves the one or more index ring release tabs from the initial position (PraIN) to the release position (PraR). The one or more index ring release tabs may be part of the syringe holder.

In one or more examples, the one or more index ring release tabs comprises a protruding surface engaging a blocking surface on the index ring in the initial position (PraIN) thereby preventing the index ring from rotating from the first rotational position (R1) to the second rotational position (R2). The one or more index ring release tabs may be configured for flexing radially inwardly by proximate movement of the one or more release members, when the cassette is secured in the auto injector. When the one or more index ring release tabs flex radially inwardly, the protruding surface normally no longer engages the blocking surface on the index ring whereby the index ring is rotatable from the first rotational position (R1) to the second rotational position (R2).

In one or more examples, the index ring is rotatable from the first rotational position (R1) to the second rotational position (R2) by movement of the needle safe cover distally.

In one or more examples, the index ring is configured for rotating from the first rotational position (R1) to the second rotational position (R2) by movement of the first needle safe cover distally when the protruding surface no longer engages the blocking surface on the index ring.

In one or more examples, the index ring is configured for rotating from the first rotational position (R1) to the second rotational position (R2) by the second index ring engaging finger on the second needle safe cover traveling inside a first secondary track section on the index ring.

In one or more examples, when the one or more release members are in the first release member position inside the cassette, further rotation of the index ring from the second rotational position (R2) to a third rotation position (R3) is prevented by the one or more release members engaging the first track in the index ring.

In one or more examples, the index ring is rotatable from the second rotational position (R2) to the third rotation position (R3) upon proximal movement of the one or more release members of the auto injector from the first release member position inside the cassette to a second release member position inside the cassette.

In one or more examples, the index ring is rotatable from the second rotational position (R2) to the third rotation position (R3) upon further proximal movement of the one or more release members of the auto injector from the first release member position inside the cassette to a second release member position inside the cassette, the second release member position being proximal of the first release member position.

In one or more examples, the index ring is configured for rotating from the second rotational position (R2) to the third second rotational position (R3) by movement of the first needle safe cover distally after proximal movement of the one or more release members of the auto injector from the first release member position inside the cassette to the second release member position inside the cassette.

In one or more examples, the index ring is configured for rotating from the second rotational position (R2) to the third second rotational position (R3) by the second index ring engaging finger on the second needle safe cover traveling inside a third secondary track section on the index ring.

In one or more examples, when the index ring is in the third second rotational position (R3), the second index ring engaging finger no longer abuts the at least one of the one or more release arms on the index ring, whereby proximal movement of the first needle safe cover and the second needle safe cover relative to the index ring to a final locked position is allowed.

In one or more examples, when the one or more release members are in the first release member position inside the cassette, further rotation of the index ring from the second rotational position (R2) to a third rotation position (R3) is prevented by the one or more release members engaging a first track in the index ring.

In one or more examples, the index ring is in the second rotational position (R2) when the first medicament component and the second medicament component are mixed if a dual chamber syringe is used.

The one or more release arms may be a separate item. Alternatively, in one or more examples, the release arms are part of the index ring. The release arms may be positioned at the proximal end of the index ring.

In one or more examples, the first needle safe cover comprises a first index ring engaging finger and the second needle safe cover comprises a second index ring engaging finger. When the index ring is in the first rotational position (R1), the first index ring engaging finger and the second index ring engaging finger may abut at least one of the one or more release arms on the index ring preventing proximal movement of the first needle safe cover and the second needle safe cover relative to the index ring.

When the index ring is in the second rotational position (R2), the first index ring engaging finger may no longer abut the at least one of the one or more release arms on the index ring whereby the one or more cover springs are configured for moving the first needle safe cover proximately relative to the second needle safe cover thereby moving the first needle safe cover into a locked cover position where further relative longitudinal movement between the first needle safe cover and the second needle safe cover is prevented.

Normally, there are two index ring engaging fingers on both the first and the second needle safe cover. They are positioned on opposite sides symmetrically.

In one or more examples, the first needle safe cover and the second needle safe cover comprises a first locking pair preventing proximal movement of the first needle safe cover relative to the second needle safe cover and a second locking pair preventing distal movement of the first needle safe cover relative to the second needle safe cover in the locked cover position. The proximal locking may be obtained by a first locking pair constituting a proximal first needle safe cover interface on the first needle safe cover and a distal second needle safe cover locking tab on the second needle safe cover. The distal locking may be obtained by a second locking pair constituting a distal first needle safe cover locking surface on the first needle safe cover and a proximal second needle safe cover locking tab on the second needle safe cover. The surfaces/parts on the first and second needle safe covers abut each other for creating the locking pairs.

In one or more examples, the index ring comprises a second track extending from a first secondary corner to a second secondary corner of a surface area of the index ring, wherein the index ring is configured for rotating from the first rotational position (R1) to the second rotational position (R2) by movement of the needle safe cover distally whereby at least one protruding part on the needle safe cover travels inside the second track thereby rotating the index ring.

In one or more examples, the index ring comprises a second track extending from a first secondary corner to a second secondary corner of a surface area of the index ring, wherein the index ring is configured for rotating from the first rotational position (R1) to the second rotational position (R2) by movement of the second needle safe cover distally whereby a second index ring engaging finger on the second needle safe cover travels inside the second track thereby rotating the index ring.

In one or more examples, the second track comprises a first secondary track section and a second secondary track section, wherein:
the first secondary track section extends from the first secondary corner in a first secondary angled direction towards a first secondary middle point;
the second secondary track section extends from the first secondary middle point to a second secondary middle point along the longitudinal direction of the cassette in a second secondary angle direction.

The first secondary angled direction may be between 20 and 70 degrees, such as between 30 and 60 degrees, such as between 35 and 55 degrees, such as between 40 and 50 degrees, such as approximately 45 degrees relative to the longitudinal direction of the cassette.

The second secondary angled direction may be between −20 and 20 degrees, such as between −10 and 10 degrees, such as approximately 0 degrees relative to the longitudinal direction of the cassette.

In one or more examples, the second track further comprises a third secondary track section, wherein the third secondary track section extends from the second secondary middle point in a third secondary angled direction towards the second secondary corner. The third secondary angled direction may be between 20 and 70 degrees, such as between 30 and 60 degrees, such as between 35 and 55 degrees, such as between 40 and 50 degrees, such as approximately 45 degrees relative to the longitudinal direction of the cassette.

In one or more examples, the second track further comprises a fourth secondary track section. The fourth secondary track section normally extends from the second secondary corner and distally. The fourth secondary track section may have a plateau track section with a fourth angled direction between −20 and 20 degrees, such as between −10 and 10 degrees, such as approximately 0 degrees relative to the longitudinal direction of the cassette.

In one or more examples, the index ring is configured for rotating from the first rotational position (R1) to the second rotational position (R2) when the cassette is inserted in the auto injector and the one or more release members comprised in the auto injector travels proximally inside the track.

In one or more examples, the index ring is rotatable from the second rotational position (R2) to the third rotation position (R3) upon proximal movement of the one or more release members of the auto injector from the first release member position inside the cassette to a second release member position inside the cassette.

In one or more examples, the index ring is configured for rotating from the second rotational position (R2) to the third second rotational position (R3) by movement of the first needle safe cover distally after proximal movement of the one or more release members of the auto injector from the first release member position inside the cassette to the second release member position inside the cassette.

In one or more examples, when the index ring is in the third second rotational position (R3), the second index ring engaging finger no longer abuts the at least one of the one or more release arms on the index ring, whereby proximal movement of the first needle safe cover and the second needle safe cover relative to the index ring to a final locked position is allowed.

In one or more examples, in the initial position (PraIN), the first needle safe cover is prevented from moving proximally relative to the second needle safe cover. Likewise, in the release position (PraR), the first needle safe cover may be allowed to move proximally relative to the second needle safe cover. The proximal movement of the first needle safe cover may allow the needle mounting blocking system/the needle safe cover parts to extend telescopically. The telescopic extension by moving the first needle safe cover proximally relative to the second needle safe cover will normally increases the distance between the proximal ends of the first needle safe cover and the second needle safe when the first needle safe cover is positioned proximally of the second needle safe cover before the telescopic extension.

Prior to securing the cassette in the auto injector, the first needle safe cover and the second needle safe cover may be allowed to move towards each other by compression of the one or more cover springs. This may facilitate mounting of the needle assembly to the cassette.

In one or more examples, the first needle safe cover comprises one or more cover locking tabs locking the first needle safe cover in the final locked position by preventing distal movement of the first needle safe cover relative to a cassette housing.

In one or more examples, the second needle safe cover comprises at least one flexible arm, the first needle safe cover comprises a first locking tab, and the one or more release arms each comprises a protruding surface release part, wherein the protruding surface release part limits the proximal movement of the first needle safe cover relative to the second needle safe cover in the initial position (PraIN) by preventing the at least one flexible arm from flexing when abutting the first locking tab. In this manner, the telescopic movement of the two needle safe cover parts are limited by the one or more release arms.

In one or more examples, upon securing the needle assembly to the syringe holder, the second needle safe cover, the first needle safe cover and the one or more release arms are configured for moving distally relative to the syringe holder. The needle safe cover parts/the needle mounting blocking system may be configured for being pushed distally when the user secures the needle assembly to the cassette.

In one or more examples, each of the second needle safe cover, the first needle safe cover and the one or more release arms moves a different distance distally relative to the syringe holder upon securing the needle assembly to the syringe holder. Each of the second needle safe cover, the first needle safe cover and the one or more release arms may this move relative to each other when they are pushed distally upon securing the needle assembly to the cassette. Alternatively, two of the parts may move longitudinally the same distance.

In one or more examples, the needle assembly further comprises a removable needle cap covering and protecting the needle prior to use, wherein after securing the needle assembly to the syringe holder and removing the needle cap, the one or more cover springs are adapted for pushing the first needle safe cover in the proximal direction relative to the second needle safe cover whereby the first needle safe cover covers the needle in a ready for use position (PnsR). Thus, the proximal end of the needle mounting blocking system is in a ready for use position (PnsR). The one or more cover springs may as stated above alternatively be substituted by a spring system inside the auto injector acting on the first needle safe cover relative to the second needle safe cover after mounting of the cassette in the auto injector.

In one or more examples, after securing the needle assembly to the syringe holder, the one or more release arms are locked relative to the syringe holder.

In one or more examples, when the one or more release arms are in the release position (PraR) after securing the needle assembly to the syringe holder and removing the needle cap, the second needle safe cover is adapted for being moved proximally by the auto injector while the one or more release arms are locked relative to the syringe holder. The proximal movement of the second needle safe cover relative to the one or more release arms may push the first needle safe cover and the second needle safe cover into positions where a telescopic movement between the second needle safe cover parts are possible. Proximal movement of the second needle safe cover may result in the first needle safe cover following suit. Alternatively, the proximal movement of the first needle safe cover may result in the second needle safe cover following suit.

In one or more examples, when the one or more release arms are in the release position (PraR) after securing the needle assembly to the syringe holder and removing the needle cap, the one or more cover springs are adapted for pushing the first needle safe cover in the proximal direction relative to the second needle safe cover thereby moving the at least one flexible arm proximally and past the protruding surface release part into a position where the at least one flexible arm flexes thereby allowing the first locking tab of the first needle safe cover to pass the at least one flexible arm pushing the first needle safe cover into an extended position relative to the second needle safe cover by means of the one or more cover springs. The proximal movement of the first needle safe cover in the above example results in the second needle safe cover following suit.

In one or more examples, when the cassette is in the ready for use position (PnsR), the one or more locking arms are in the release position (PraR) and the needle safe cover parts/the needle mounting blocking system are/is in a locked cover position where further relative longitudinal movement of the first needle safe cover relative to the second needle safe cover is prevented. The position where the first needle safe cover is locked relative to the second needle safe cover does not prevent the two parts from moving together as one combined needle safe cover.

In one or more examples, the first needle safe cover comprises a second locking tab positioned distally of the first locking tab, and the second needle safe cover comprises a stop surface positioned proximally of the flexible arm, wherein in the locked cover position, the second locking tab abuts the flexible arm preventing distal movement of the first needle safe cover relative to the second needle safe cover and the first locking tab abuts the stop surface preventing proximal movement of the first needle safe cover relative to the second needle safe cover. The first needle safe cover is thereby locked relative to the second needle safe cover.

In one or more examples, the first needle safe cover is movable between a plurality of longitudinal positions relative to the syringe holder, the plurality of longitudinal positions including:
a ready for use position (PnsR);
a medicament delivery position (PnsD) where the needle is exposed, wherein the medicament delivery position (PnsD) is distally of the ready for use position (PnsR);
a final locked position (PnsFL) where further longitudinal movement of the first needle safe cover is prevented, wherein the final locked position (PnsFL) is proximal of the medicament delivery position (PnsD).

The ready for use position protects the user and/or a medical practitioner delivering the medicament to a patient from accidental contact with the needle prior to initiating medicament delivery. In the final locked position, the injection needle may be covered after medicament delivery thereby preventing unintentional contact with the needle after medicament delivery.

In one or more examples, the first needle safe cover covers the needle in the ready for use position (PnsR).

In one or more examples, the final locked position (PnsFL) is proximal of the ready for use position (PnsR).

In one or more examples, distal movement of the one or more release members after medicament delivery rotates the index ring back to the first rotational position (R1), whereby the proximal end of the needle mounting blocking system and/or the first needle safe cover is longitudinally locked in the final locked position (PnsFL). The index ring may thereby assist in securing the needle mounting blocking system and/or the first needle safe cover in the final locked position after medicament delivery.

In one or more examples, the second needle safe cover further functions as a needle mounting blocking system configured for moving between:
a locked position (PnsL) preventing piercing of the seal by the needle prior to securing the cassette in the auto injector; and
a needle assembly connecting position (PnsC) where piercing of the seal by the needle is allowed after securing the cassette in the auto injector.

The movement of the second needle safe cover may be configured to be controlled by the auto injector after securing the cassette in the auto injector. It is therefore obtained that any premature contact between an injection needle and the medicament inside the syringe compartment is prevented. It is simply not possible for a user to secure a needle to the syringe and thereby bring the injection needle into fluid contact with the medicament before the user has secured the cassette in the auto injector. This prevents situations, where the user would connect a needle assembly to a cassette, most likely piercing the seal, with the purpose of being prepared for a fast injection at a later point in time

In one or more examples, the needle assembly comprises a removable needle cap, wherein removal of the needle cap when the proximal end of the needle mounting blocking system is in the medicament delivery position (PnsD) will allow the proximal end of the needle mounting blocking system to move proximally.

In one or more examples, the plurality of longitudinal positions, which the proximal end of the needle mounting blocking system is moveable between, further comprises:
a medicament delivery position (PnsD) where the needle is exposed;
a final locked position (PnsFL) where further longitudinal movement of the needle mounting blocking system is prevented,
   wherein the medicament delivery position (PnsD) and the final locked position (PnsFL) are at the same longitudinal position. The needle mounting blocking system thereby stays in the medicament delivery position when being locked after use.

In one or more examples, after medicament delivery, distal movement of the one or more release members in the auto injector is configured for rotating the index ring back to the first rotational position (R1), whereby the proximal end of the needle mounting blocking system is longitudinally locked in the final locked position (PnsFL).

In one or more examples, when the index ring is in the second rotational position (R2), the one or more cover springs moves the first needle safe cover proximately relative to the second needle safe cover into a locked cover position where further relative longitudinal movement between the first needle safe cover and the second needle safe cover is prevented by one or more locking means.

In one or more examples, the one or more locking means comprises a first locking pair, wherein the first needle safe cover and the second needle safe cover comprises the first locking pair preventing proximal movement of the first needle safe cover relative to the second needle safe cover.

In one or more examples, the one or more locking means comprises a second locking pair, wherein the first needle safe cover and the second needle safe cover comprises the second locking pair preventing distal movement of the first needle safe cover relative to the second needle safe cover.

In one or more examples, the needle safe cover is configured for moving between:
a locked position (PnsL) preventing piercing of the seal by the needle prior to securing the cassette in the auto injector; and
a needle assembly connecting position (PnsC) where piercing of the seal by the needle is allowed after securing the cassette in the auto injector.

When the index ring is in the first rotational position (R1), the needle safe cover may be in to the locked position (PnsL) and wherein when the index ring is in the second rotational position (R2), the needle safe cover may be movable the needle assembly connecting position (PnsC) by movement of the needle safe cover distally.

In one or more examples, the needle safe cover according to the third aspect comprises:
a needle safe cover; and
a second needle safe cover, wherein the first needle safe cover is displaceable relative to the second needle safe cover along the longitudinal direction.

In the initial position (PraIN), the first needle safe cover and the second needle safe cover may be separated by at the most a first separation distance. In the release position (PraR), the first needle safe cover and the second needle safe cover may be allowed to move relatively into a second separation distance being larger than the first separation.

In one or more examples, the cassette according to any of the aspects with a two-part safe further comprises one or more cover springs biased between the first needle safe cover and the second needle safe cover. The first needle safe cover may comprise a first index ring engaging finger and the second needle safe cover comprises a second index ring engaging finger. When the index ring is in the first rotational position (R1), the first index ring engaging finger and the second index ring engaging finger on the second needle safe cover normally abut at least one of the one or more release arms on the index ring preventing proximal movement of the first needle safe cover and the second needle safe cover relative to the index ring. When the index ring is in the second rotational position (R2), the first index ring engaging finger normally no longer abuts the at least one of the one or more release arms on the index ring whereby the one or more cover springs are configured for moving the first needle safe cover proximately relative to the second needle safe cover thereby moving the first needle safe cover into a locked cover position where further relative longitudinal movement between the first needle safe cover and the second needle safe cover is prevented.

In one or more examples, the first needle safe cover and the second needle safe cover comprises a first locking pair preventing proximal movement of the first needle safe cover relative to the second needle safe cover and a second locking pair preventing distal movement of the first needle safe cover relative to the second needle safe cover in the locked cover position.

In one or more examples, the first needle safe cover comprises one or more housing locking parts locking the first needle safe cover in the final locked position by preventing distal movement of the first needle safe cover relative to a cassette housing.

In one or more examples, the first needle safe cover comprises one or more housing locking parts locking the first needle safe cover in the final locked position by preventing distal movement of the first needle safe cover relative to a cassette housing.

In one or more examples, the one or more release arms are separate parts not forming part of the index ring, the second needle safe cover and the first needle safe cover.

Disclosed herein in a fourth aspect is further a system for use in an auto injector for administering a medicament. The system comprises a cassette according to the first aspect or the second aspect or to the third aspect and a syringe containing the medicament. The syringe may comprise a seal at a proximal end of the syringe, the seal being configured for being pierced by a distal end of a needle to obtain a fluid communication between the medicament in the syringe and the needle. Prior to securing the cassette in the auto injector, the first needle safe cover prevents the distal end of the needle from piercing the seal. The proximal end of the needle is configured for piercing the seal of the syringe and a proximal end of the needle is configured for piercing an injection site.

The system may further comprise a needle assembly comprising the needle, a needle assembly adaptor for securing the needle assembly to the syringe holder, and a removable needle cap covering and protecting the needle.

The syringe may comprise a syringe compartment containing the medicament. The compartment may be a one-chamber compartment. Alternatively, the syringe compartment may be a two-chamber compartment comprising a first sub-compartment containing a first medicament component and a second sub-compartment containing a second medicament component, wherein a second stopper is separating the two sub-compartments. The medicament in the first medicament component and the second sub-compartment may be mixed prior to medicament delivery by proximal movement of second stopper.

The syringe may be a 1-3 mL syringe, such as a 1 mL, a 2.25 mL, or a 3 ml syringe. Examples include a 3 mL liquid one-chamber cartridge and a 1 mL dual-chamber cartridge. When reference is made to a 1 mL dual-chamber cartridge, 1 mL may refer to the resulting reconstituted liquid medicament to be injected, i.e. the total amount of medicament after mixing. The syringe may further have a sleeve at the distal end of the syringe for easy mounting in the syringe holder.

In one or more examples, the system further comprising the auto injector, wherein the auto injector is configured for receiving the cassette.

The auto injector may comprise one or more release members configured for moving into the cassette when the cassette has been received in the auto injector.

In one or more examples, the needle assembly further comprises a needle assembly shield configured for being in a plurality of positions including:
an initial delivery position, where the needle assembly shield of the needle assembly covers the needle;
a medicament delivery position, where the needle is exposed;
a final locked position, where the needle assembly shield of the needle assembly covers the needle.

Disclosed herein in a fifth aspect is an auto injector for administering a medicament, the auto injector being configured for receiving any of the cassettes according to the first aspect or the second aspect or the third aspect and/or configured for receiving a system according to the fourth aspect without requiring modifications. This allows the auto injector to be used with different types of cassettes without there being any need to modify the auto injector. The needed modifications depending on medicament type requirements, difference in needle assembly design and the likes, are all included as modifications to the individual cassettes. Alternative, small modifications to e.g. the electronic parts of the auto injection may be imagined.

Disclosed herein in a sixth aspect is a system comprising an auto injector for administering a medicament, and a cassette according to the first aspect or the second aspect or the third aspect. The system may further comprise a needle assembly comprising the needle, a needle assembly adaptor for securing the needle assembly to the syringe holder, and a removable needle cap covering and protecting the needle, wherein prior to securing the cassette in the auto injector, the needle mounting blocking system prevents a distal end of the needle from piercing the seal. The distal end of the needle is for penetrating the septum. The proximal end of the needle is for penetrating an injection site during medicament delivery.

### BRIEF DESCRIPTION OF THE DRAWINGS

Various examples are described hereinafter with reference to the figures. Like reference numerals refer to like elements throughout. Like elements will, thus, not be described in detail with respect to the description of each figure. It should also be noted that the figures are only intended to facilitate the description of the examples. They are not intended as an exhaustive description of the claimed invention or as a limitation on the scope of the claimed invention. In addition, an illustrated example needs not have all the aspects or advantages shown. An aspect or an advantage described in conjunction with a particular example is not necessarily limited to that example and can be practiced in any other examples even if not so illustrated, or if not so explicitly described.

FIG. 1 shows an exploded view of the different parts in a first example of the cassette.

FIGS. 2A-I show the separate part of the first cassette.

FIGS. 3A-L show the first cassette during mounting of the needle assembly and medicament delivery in a partly cut-through perspective view.

FIGS. 4A-L show the first cassette during mounting of the needle assembly and medicament delivery in a cut-through view.

FIGS. 6A-H show a second cassette during mounting of the needle assembly and during medicament delivery.

FIG. 7 shows an exploded view of the different parts in the second example of the cassette shown in FIGS. 6A-H.

FIGS. 8A-F show a third cassette before connecting to a needle assembly, during connecting to the needle assembly, and after removal of the needle assembly.

FIGS. 9A-H show the third cassette during mounting of the needle assembly and during medicament delivery.

FIGS. 10A-C show a dual chamber syringe for use with any of the shown examples of the cassette.

FIG. 11 shows an exploded view of the different parts in a fourth example of the cassette.

FIGS. 12A-H show the separate part of the fourth first cassette.

FIGS. 13A-K show the forth cassette during mounting of the needle assembly and medicament delivery in a cut-through view.

FIGS. 14A-G show a close-up of the interaction between the first needle safe cover, the second needle safe cover, the index ring and the syringe holder during the process of mounting a needle assembly to the syringe holder.

FIGS. 15A-B show an auto injector configured for receiving a cassette according to the first or second example, where FIG. 15A shows the assemble injector and FIG. 15B shows a view of the surface in the auto injector in contact with the cassette.

FIG. 16 shows an exploded view of the auto injector of FIGS. 15A-B.

### DESCRIPTION OF EXAMPLES

Exemplary examples will now be described more fully hereinafter with reference to the accompanying drawings. In this regard, the present examples may have different forms and should not be construed as being limited to the descriptions set forth herein. Accordingly, the examples are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

In the drawings, thicknesses of a plurality of layers and areas are illustrated in an enlarged manner for clarity and ease of description thereof. When a layer, area, element, or plate is referred to as being “on” another layer, area, element, or plate, it may be directly on the other layer, area, element, or plate, or intervening layers, areas, elements, or plates may be present therebetween. Conversely, when a layer, area, element, or plate is referred to as being “directly on” another layer, area, element, or plate, there are no intervening layers, areas, elements, or plates therebetween. Further when a layer, area, element, or plate is referred to as being “below” another layer, area, element, or plate, it may be directly below the other layer, area, element, or plate, or intervening layers, areas, elements, or plates may be present therebetween. Conversely, when a layer, area, element, or plate is referred to as being “directly below” another layer, area, element, or plate, there are no intervening layers, areas, elements, or plates therebetween.

The spatially relative terms “lower” or “bottom” and “upper” or “top”, “below”, “beneath”, “less”, “above”, and the like, may be used herein for ease of description to describe the relationship between one element or component and another element or component as illustrated in the drawings. It will be understood that the spatially relative terms are intended to encompass different orientations of the device in use or operation, in addition to the orientation depicted in the drawings. For example, in the case where a device illustrated in the drawings is turned over, elements described as being on the “lower” side of other elements, or “below” or “beneath” another element would then be oriented on “upper” sides of the other elements, or “above” another element. Accordingly, the illustrative term “below” or “beneath” may include both the “lower” and “upper” orientation positions, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below, and thus the spatially relative terms may be interpreted differently depending on the orientations described.

Throughout the specification, when an element is referred to as being “connected” to another element, the element is “directly connected” to the other element, or “electrically connected” to the other element with one or more intervening elements interposed therebetween.

The terminology used herein is for the purpose of describing particular examples only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that, although the terms “first,” “second,” “third,” and the like may be used herein to describe various elements, these elements should not be limited by these terms. These terms are only used to distinguish one element from another element. Thus, “a first element” discussed below could be termed “a second element” or “a third element,” and “a second element” and “a third element” may be termed likewise without departing from the teachings herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

Exemplary examples are described herein with reference to cross section illustrations that are schematic illustrations of idealized examples, wherein like reference numerals refer to like elements throughout the specification. As such, variations from the shapes of the illustrations as a result, for example, of manufacturing techniques and/or tolerances, are to be expected. Thus, examples described herein should not be construed as limited to the particular shapes of regions as illustrated herein but are to include deviations in shapes that result, for example, from manufacturing. For example, a region illustrated or described as flat may have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims. Some of the parts which are not associated with the description may not be provided in order to specifically describe exemplary examples of the present disclosure.

By proximal end/proximal surface is meant the end/surface part pointing towards the needle, i.e. the end of the cassette, which is closest to the injection site during medicament delivery. By distal end/distal surface is meant the end/surface part pointing in a direction away from the needle, i.e. opposite the end of the cassette, which is closest to the injection site during medicament delivery.

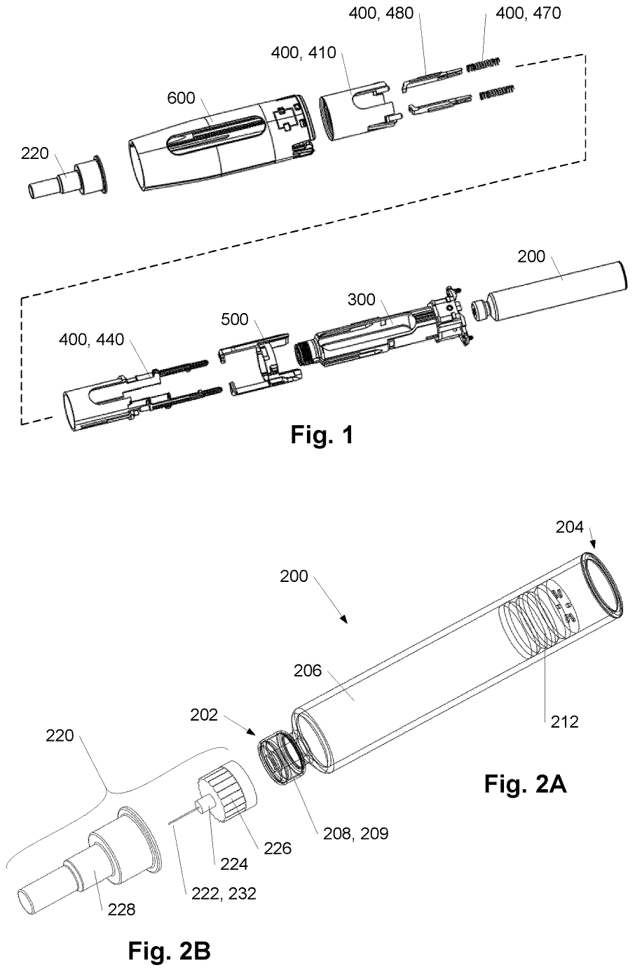

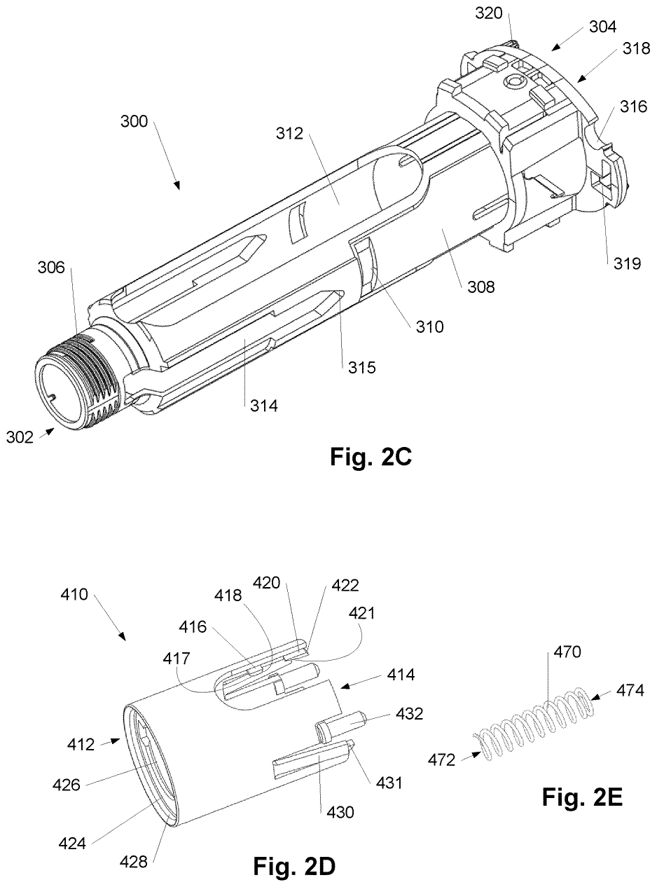

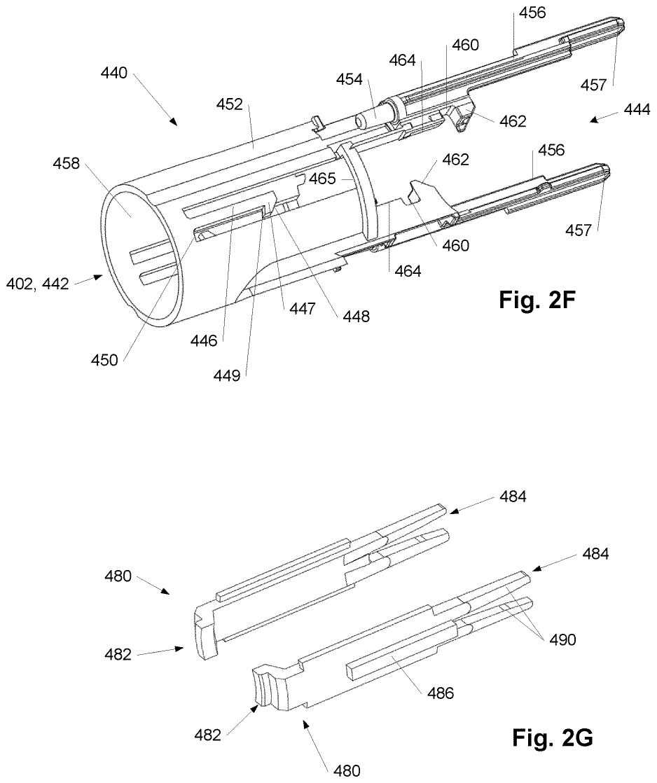

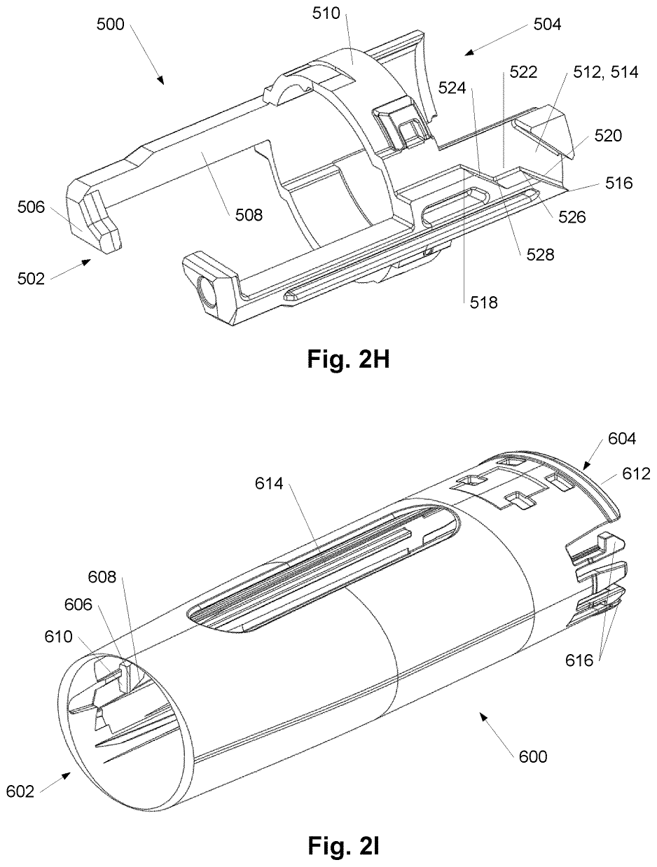

FIG. 1 shows an exploded view of the different parts in a first example of the cassette 100, whereas FIGS. 2A-I show the separate parts of the cassette as shown in FIG. 1 as separate parts enlarged for a clearer view. The cassette 100 comprises a syringe holder 300 for receiving a syringe 200, a first needle safe cover 410, a second needle safe cover 440, release arms 480, cover springs 470, and an index ring 500. The first needle safe cover 410, the second needle safe cover 440, the release arms 480, and the cover springs 470 are part of a needle safe cover assembly 400. The needle safe cover assembly 400 is a telescopically assembly, where the distance between the two needle safe cover parts 410, 440 can change. The first needle safe cover 410 and the second needle safe cover 440 extends around the syringe holder 300.

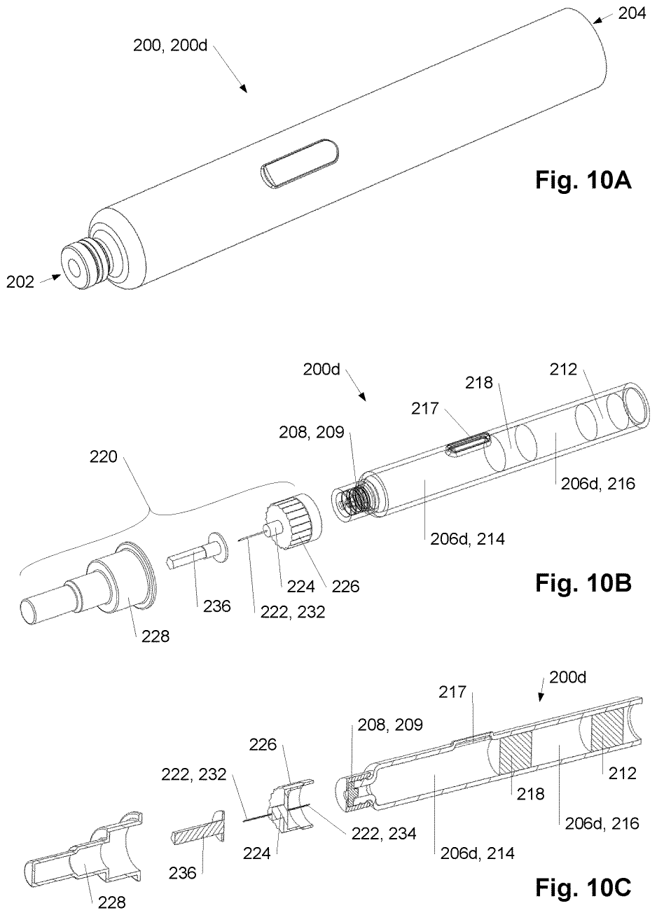

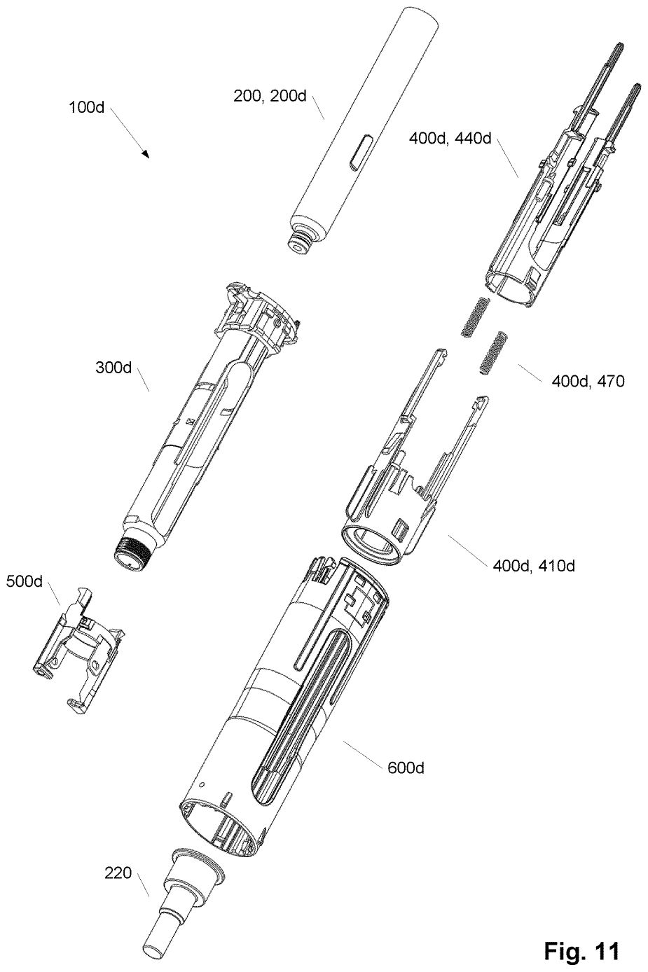

The syringe 200 shown enlarged in FIG. 2A and FIG. 10A extends from a proximal end 202 to a distal end 204. The medicament is contained inside a syringe compartment 206, 206d extending between the proximal end 204 and the distal end 204 of the syringe 200. At the proximal end of the syringe 200 is a seal/septum 208, which-when pierced by a needle 222 in a needle assembly 220-constitutes a syringe outlet 209 for the medicament to exit the syringe compartment 206, 206d. Inside the syringe compartment 206, 206d is a first stopper 212, which pushes medicament out of the syringe outlet 209 when moving in the proximal direction. Normally, the first stopper 212 is pushed in the proximal direction by means of a plunger rod 1018 included in an auto injector 1000 into which the cassette 100 with the syringe 200 is mounted.

A single chamber syringe 200 as shown in FIG. 2A may be used. Alternatively, a dual chamber syringe 200d as shown in FIGS. 10A-C may also be used.

In the dual chamber syringe 200d, the syringe compartment 206d comprises two sub-compartments 214, 216. One sub-compartment 216 may comprise a liquid medicament component and the other a solid/powder component 214. Such cartridge may be referred to as a liquid-powder two compartment cartridge. Alternatively, two different liquid components may be found one liquid in each a sub-compartment 214, 216. Such syringe 200d may be referred to as a liquid-liquid two compartment cartridge. The two sub-compartments 214, 216 may be separated by a second stopper 218. A liquid medicament component is normally positioned between the two stoppers 212, 218. When moving the first stopper 212 proximally, the second stopper 218 is normally moved to a by-pass region 217, where the two medicament components are allowed to come in contact with each other and mix. In the by-pass region 217, the liquid component found in the sub-compartment 216 between the two stoppers 212, 218, is allowed to pass proximately of the second stopper 218 thereby entering into the most proximally positioned sub-compartment 214 for mixing with the solid/powder/liquid component in this sub-compartment 214. Further movement of the first stopper 212, pushes the second stopper 218 and the mixed medicament proximately for medicament delivery.

As an alternative to a by-pass region and a mixing of the two medicament components, a sequential delivery may also be envisioned. In such scenario, the cartridge will normally be a liquid-liquid two compartment cartridge, where medicament in the most proximal positioned sub-compartment is delivered first and medicament in the most distally positioned sub-compartment is delivered second. For providing access to the medicament in the most distally positioned sub-compartment, one or more interior needles for penetrating the second stopper may be included in the cartridge.

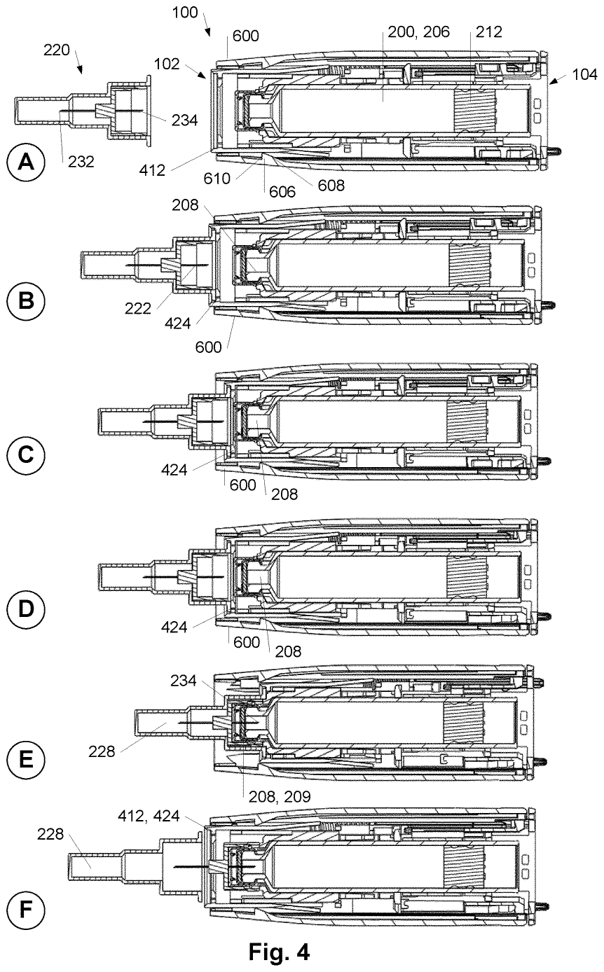

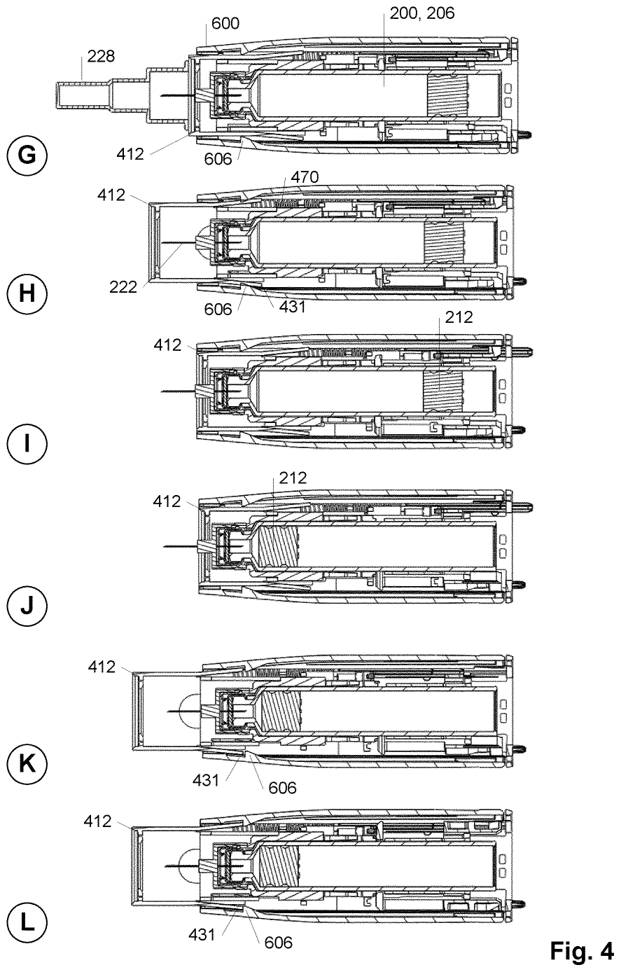

The needle assembly 220 shown in FIG. 2B comprises a needle 222 and a needle hub 224 through which the needle 222 extends. The needle 222 protrudes from both ends of the needle hub 224. The proximal end 232 of the needle 222 is inserted into the injection site, i.e. into the patient, during medicament delivery. When the needle assembly 220 is secured to the syringe 220, the distal end 234 of the needle 222 (see FIG. 4A) penetrates the septum 208 in the syringe 200, 200d whereby the syringe outlet 209 is established. The needle assembly 220 further comprises a needle assembly adaptor 226 by which the needle assembly 220 may be secured to the syringe holder 300. Prior to use, the needle 222 is protected by a needle cap 228, e.g. a rigid needle shield.

The syringe holder 300 shown in FIG. 2C extends between a proximal end 302 and a distal end 304. Normally, the syringe holder 300 is longitudinally locked relative to the cassette housing 600 shown in FIG. 21. At the proximal end 302 of the syringe holder 300 is a needle assembly locking part 306 for connecting the needle assembly 220 to the syringe holder 300. The needle assembly locking part 306 on the syringe holder 300 connects to the needle assembly adaptor 226 on the needle assembly 220 by a threaded connection. The connection between the two parts could also be any other type of connection, which would allow for securing the needle assembly 220 to the syringe holder 300. A press-fit connection is an example of such. Further, a press fit connection might be a result of a special needle in combination with the threaded interface such that the special needle effectively slides over the thread without the need for user induced rotation. At the end of the sliding movement, the thread and the interface inside the needle hub may ensure that the special needle cannot be pulled off again easily. As an alternative, a bayonet fixation principle could also be envisioned. For the bayonet fixation principle, a user normally performs a combination of pressing of the needle assembly against the syringe holder possibly combined with a minor rotation of the needle assembly relative to the syringe holder.

The syringe holder 300 comprises an elongated tubular part 308 inside which the syringe 200, 200d is found when the syringe 200, 200d is mounted in the syringe holder 300. The elongated tubular part 308 has a number of openings including two windows 312 for inspection of the syringe 200, 200d. The elongated tubular part 308 further has two release arm receiving channels 314 each for receiving a release arm 480 (see FIG. 2G for the release arm). The release arm receiving channels 314 are closed at the distal end providing a release arm blocking surface 315 preventing further distal movement of the release arm 480 once the distal end 484 of the release arm 480 abuts the release arm blocking surface 315.

At the distal end 304 of the syringe holder 300 is a distal ring part 316. The distal ring part 316 comprises a plunger opening 318 through which a plunger rod 1018 from the auto injector 1000 can pass. The plunger opening 318 also allows for inserting the syringe 200, 200d into the syringe holder 300. The distal ring part 316 further has two release member openings 319 through which release members 1006 of the auto injector can pass after assembly of the cassette 100 in the auto injector 1000.

Along the elongated tubular part 308 are further index ring receiving openings 310 for receiving at least a part of an index ring locking protrusions 506 of the index ring 500. The index ring locking protrusion 506 is allowed to rotate in the index ring receiving opening 310.

The first needle safe cover 410 shown in FIG. 2D extends between a proximal end 412 and a distal end 414 and has a tubular shape. At the proximal end 412 is an opening 426 in a proximally positioned surface plate 424 through which the needle assembly 220 may be attached to the syringe holder 300. When connecting the needle assembly 220 to the syringe holder 300, the needle cap 228 in the needle assembly 220 abuts the proximal surface of the surface plate 424 of the first needle safe cover 410. When the first needle safe cover 410 is in an unlocked initial position Pns1IN, the user may thus push the first needle safe cover 410 distally relative to the syringe holder 300 by pushing the needle assembly 220 distally. The first needle safe cover 410 extends around the second needle safe cover 440 at least in part. The distal surface of the surface plate 424 of the first needle safe cover 410 thereby comes in contact with the proximal end 442 of the second needle safe cover 440 when the first needle safe cover 410 is pushed distally prior to the two needle safe cover parts being in an extended locked position Pns 12L.

The surface plate 424 is positioned a short distance distal of the proximal end 412 of the first needle safe cover 410. A narrow inner support surface 428 is thereby created proximately of the surface plate 424 on the inner surface of the tubular first needle safe cover 410. The inner support surface 428 may be used for supporting the needle assembly 220 prior to connecting the needle assembly 220 to the syringe holder 300. In this manner, the needle assembly 220 and the cassette 100 may be delivered as a combined kit, in which the needle 222 not yet has penetrated the septum 208.

The first needle safe cover 410 comprises a first locking tab 416. The first locking tab 416 has a proximal sloping surface 417. The first needle safe cover 410 further comprises a second locking tab 420. Both locking tabs 416, 420 are positioned on the inner surface of the first needle safe cover 410. The first locking tab 416 is proximal of the second locking tab 420. The second locking tab 417 likewise has a proximal sloping surface 421. The sloping surfaces 417, 421 facilitates that the first needle safe cover 410 can move proximately relative to the second needle safe cover 440 by allowing a flexible arm 446 on the second needle safe cover 440 to move along and pass the first sloping surface 417 and the second sloping surface 421. The distal surface 418 of the first locking tab 416 and the distal surface 422 of the second locking tab 420 prevent distal movement of the first needle safe cover 410 relative to the second needle safe cover 440 once the flexible arm 446 has moved passed either of the two locking tabs 416, 420. The distal locking tab surfaces 418, 422 are normally approximately perpendicular to the longitudinal direction of the cassette 100 or sloping in a direction preventing proximal movement of the flexible arm 446 once it is positioned such that it abuts the distal locking tab surfaces 418, 422.

The first needle safe cover 410 comprises two cover locking tabs 430 projecting distally and radially outwards from the tubular cover. The distal end 431 of each cover locking tab 430 rests against a housing locking protrusion 606 on the inside of the cassette housing 600 (see cassette housing in FIG. 21). The cover locking tabs 430 are flexible allowing the two distal ends 431 to be flexed radially inwardly towards each other. This allows the first needle safe cover 410 to be pushed proximally to an extended position relative to the cassette housing 600 after medicament injection. The cover locking tabs 430 move along a sloping surface 608 on the housing locking protrusion 606 when the first needle safe cover 410 moves to the extended position relative to the cassette housing 600. The proximal surface 610 of the housing locking protrusion 606 prevents the cover locking tabs 430 from moving distally once it has been moved to a position proximally of the housing locking protrusion 606. This locks the first needle safe cover 410 in an extended position where the needle 222 is covered by the first needle safe cover 410 after medicament delivery.

At the distal end 414 of the first needle safe cover 410 are further found two distally extending pins 432. Around each pin 432 is the proximal end 472 of the cover spring 470 (see enlarged view of the cover spring in FIG. 2E). The distal end 474 of each cover spring 470 extends around a proximally extending pin 454 on the second needle safe cover 440. Each pin 432, 454 support a cover spring 470, but instead of pins for support, other supports means could be used, such as e.g. a channel protruding inwards or an outer surface on the cover parts 410, 440. Further, two sets of pins/spring are shown in the figures, but the cassette could also be constructed using only one set of pins/spring or just using a single spring.

The second needle safe cover 440 is shown in an enlarged view in FIG. 2F. The second needle safe cover 440 extends between a proximal end 442 and a distal end 444. The second needle safe cover 440 has a tubular part 452. The first needle safe cover 410 extends around the tubular part 452 of the second needle safe cover 440 at least in part. The flexible arm 446 extends from the proximal end 442 of the needle safe cover 440 in the distal direction. The distal end 447 of the flexible arm 446 has a sloping surface 448, which is the surface in contact with the sloping surfaces 417, 421 on the locking tabs 416, 420 of the first needle safe cover 410 when the two needle safe cover parts 410, 440 are moved away from each other. Once either of the locking tabs 416, 420 has moved pass the flexible arm 446 in the proximal direction, distal backwards movement of the locking tabs 416, 420 is prevented by the proximal stop surface 449 on the flexible arm 446. When both second locking tab 420 has moved to a position proximal of the distal end 447 of the flexible arm 446, a stop surface 450 prevents further telescopic separation of the two needle safe cover parts 410, 440 when the first locking tab 416 abuts the stop surface 450.

When the release arms 480 are in an initial position PraIN relative to the syringe holder 300, the movement of the two needle safe cover parts 410, 440 telescopically away from each other is prevented by the release arms 480. In the initial position PraIN, the release arms 480 prevent the flexible arms 446 from flexing, and thereby prevent that the locking tabs 416, 420 of the first needle safe cover 410 can move proximally pass the sloping surface 448 of the flexible arm 446.

The tubular part 452 has an inspection opening 458 for inspection of the syringe 200, 200d. The flexible arm 446 are positioned inside the inspection opening 458. Extending distally from the tubular part 452 are distally extending pins 456. The proximally extending pin 454 around which the cover spring 470 is positioned, extends from a proximal end surface of the distally extending pin 456. The second needle safe cover 440 is normally symmetrically with a distally extending pin 456 on both sides of the second needle safe cover 440 and inspection openings 458 on both sides as well.

The distal end 457 of the distally extending pin 456 interacts with the auto injector 1000, possibly by activating a sensor informing the auto injector 1000 that a cassette 100 has been secured in the auto injector 1000.

The distally extending pin 456 comprises a first locking recess 460 and a safe cover locking protrusion 462. The safe cover locking protrusion 462 is positioned distally of the first locking recess 460. The first locking recess 460 and safe cover locking protrusion 462 interacts and couples with the index ring 500. The index ring 500 (see FIG. 2H) comprises an index ring locking protrusion 506, which is secured inside the first locking recess 460 prior to securing the cassette 100 in the auto injector. When the index ring locking protrusion 506 is positioned fully in the first locking recess 460, the second needle safe cover 440 is longitudinally locked to the index ring 500. Since the index ring 500 is normally longitudinally locked to the syringe holder 300, the second needle safe cover 440 is longitudinally locked to the syringe holder 300 when the index ring locking protrusion 506 is positioned fully in the first locking recess 460.

The index ring 500 is rotatable relative to the second needle safe cover 440. The rotation of the index ring 500 is controlled by movement of one or more release members 1006 in the auto injector 1000 into/out of the cassette 100. When the index ring 500 is rotated from an initial first rotational position R1 to a second rotation position R2, the index ring locking protrusion 506 moves to a position where it is no longer prevents the second needle safe cover 440 from moving distally. In the second rotational position R2, the second needle safe cover 440 is still prevented from moving proximally by the safe cover locking protrusion 462. The safe cover locking protrusion 462 extends further radially than the surface 464 of the distally extending pin 456 found on the proximal side of the first locking recess 460. In some examples, there is no difference between the first locking recess 460 and the surface 464. In the second rotational position R2, the needle assembly 220 can be secured to the syringe holder 300, since the second needle safe cover 440 can be pushed distally.

Further rotational movement of the index ring 500 from the second rotational position R2 to a third rotational position R3 moves the index ring locking protrusion 506 completely out of contact with the safe cover locking protrusion 462 and first locking recess 460. In the third position R3, the second needle safe cover 440 is allowed to move proximately relative to the syringe holder 300. Normally, the auto injector 1000 rotates the index ring 500 to the third rotational position R3 when the medicament delivery is initiated by proximal movement of the plunger rod 1018 in the auto injector.

When the user removes the cassette 100 and auto injector 1000 system from the injection site, a needle safe cover spring system 1026 in the auto injector 1000 pushes the second needle safe cover 440 proximately into an extended position. After medicament delivery, the auto injector 1000 retracts the one or more release members 1006. The retraction of the one or more release members 1006 rotates the index ring 500 from the third rotational position R3 to the first rotational position R1. When the index ring 500 has rotated to the first rotational position R1, the cassette is in a final locked position PnsFL from where the two needle safe cover parts 410, 440 are prevented from moving distally. This is due to the safe cover locking protrusion 462 now being proximal of the index ring locking protrusion 506, which prevents distal movement of the second needle safe cover 440.

The second needle safe cover 440 further comprises a proximal end surface 465, which the first needle safe cover 410 abuts when the needle assembly 220 is being secured to the syringe holder 300.

The release arms 480 shown in FIG. 2G each extend between a proximal end 482 and a distal end 484. At the distal end 484 of each release arm 480 are two flexible fingers 490, which can flex towards each other. The release arm 480 is positioned in the release arm receiving channel 341 in the syringe holder 300. When the release arms 480 move distally from an initial position PraIN to a release position PraR inside the release arm receiving channel 341, the flexible fingers 490 on each release arm 480 are forced towards each other by the narrowing of the release arm receiving channel 341. The release arm receiving channels 314 are closed at the distal end providing a release arm blocking surface 315 preventing further distal movement of flexible fingers 490 once they abut the release arm blocking surface 315. At the proximal end 482 of the release arm 480, the release arm 480 bends towards the centre of the cassette 100 as seen when the release arm 480 is positioned in the release arm receiving channel 341. Instead of two separate release arms 480, a release ring, where a tubular ring part connects the release arms could also be imagined. Only using one release arm is also an option.

A protruding surface release part 486 on the release arm 480 is found on the surface side pointing outwards when the release arm 480 is positioned in the release arm receiving channel 341. In the initial position PraIN, the protruding surface release part 486 is positioned distally compared to when it is in the release position PraR. In the initial position PraIN, the protruding surface release part 486 prevents proximal movement of the first needle safe cover 410 since the flexible arm 446 on the second needle safe cover 440 cannot flex to allow passage of the first locking tab 416 on the first needle safe cover 410. When the release arms 480 are moved to the release position PraR, the flexible arm 446 on the second needle safe cover 440 can flex thereby allowing the first needle safe cover 410 to be pushed into an extended position relative to the second needle safe cover 440 by the pressure from the cover springs 470 between the two needle safe covers 410, 440.

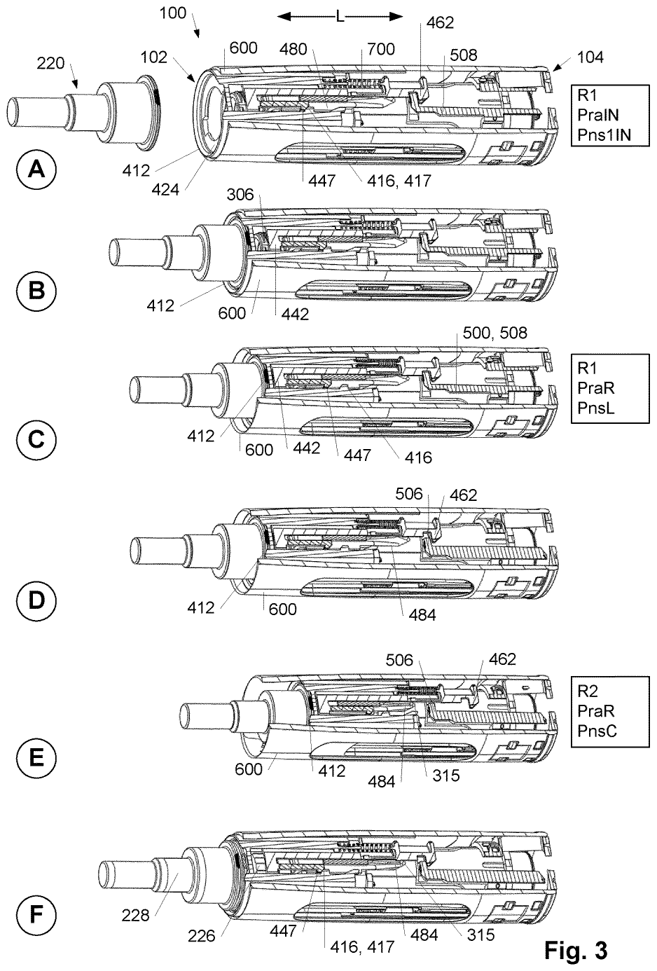

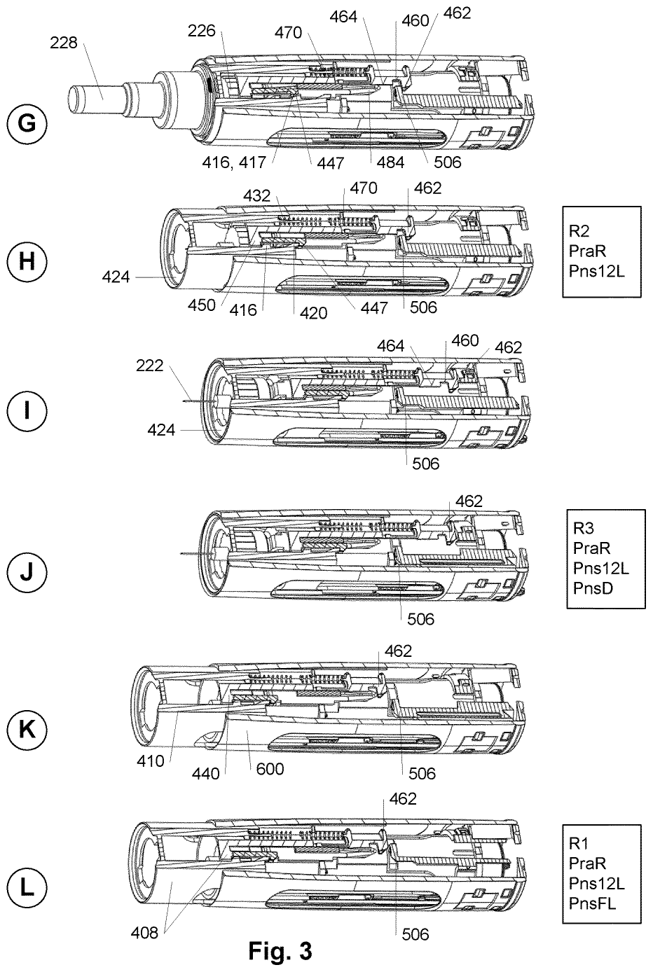

The index ring 500 shown in FIG. 2H extends from a proximal end 502 to a distal end 504. The index ring 500 is positioned around the syringe holder 300 and is normally longitudinally locked to the syringe holder 300. The index ring 500 is rotationally movable relative to the syringe holder 300 between a plurality of rotational positions including at least a first rotational position R1 in which the second needle safe cover 440 is prevented from moving distally, and a second rotational position R2 in which the second needle safe cover 440 is distally moveable. The rotational positions are illustrated in FIGS. 3A-L and FIGS. 4A-L.

The index ring 500 has two index ring locking protrusions 506 positioned each at the distal end of an index ring arm 508. The release function of the index ring locking protrusions 506 are described above.

The index ring 500 also has a tubular index ring part 510 extending around the syringe holder 300. The index ring 500 also comprises a surface area 512 partly constituting a part of the tubular index ring part 510. The surface area 512 comprises a track 514 extending from a first corner 516 of the surface area 512 to a second corner 518 of the surface area 512. The track 514 extends from the first corner 516 in an angled direction towards the second corner 518. The angled direction is normally between 20 and 70 degrees, such as between 30 and 60 degrees, such as between 35 and 55 degrees, such as between 40 and 50 degrees, such as approximately 45 degrees relative to the longitudinal direction of the cassette.

In the track 514 shown in FIG. 2H, the track 514 comprises a first track section 520, a second track section 522 and a third track section 524. The first track section 520 extends from the first corner 516 in a first angled direction towards a first middle point 526, the second track section 522 extends from the first middle point 526 to a second middle point 528 along the longitudinal direction of the cassette in a second angled direction, and the third track section 524 extends from the second middle point 528 in a third angled direction towards the second corner 518. The first and/or third angled directions are normally sloping directions, with an angle between 20 and 70 degrees, such as between 30 and 60 degrees, such as between 35 and 55 degrees, such as between 40 and 50 degrees, such as approximately 45 degrees relative to the longitudinal direction of the cassette. The second track section 522 is normally a plateau track section with a second angled direction between −20 and 20 degrees, such as between −10 and 10 degrees, such as approximately 0 degrees relative to the longitudinal direction of the cassette.

The rotational position of the index ring locking protrusion 506 and thereby the rotational position of the index ring 500 relative to the second needle safe cover 440 is controlled by one or more release members 1006 in the auto injector 1000 when the cassette 100 is secured in the auto injector 1000. More specifically, the index ring 500 is configured for rotating from the first rotational position R1 to the second rotational position R2 and again to the third rotation position R3 when the cassette 100 is inserted in the auto injector 1000 and a track-guide protrusion 1008 on the one or more release members 1006 travels proximally inside the track 514. When the track-guide protrusion 1008 travels distally again, the index ring 500 rotates back from the third rotational position R3 to the second rotational position R2 and then the first rotational position R1. Thus, by retrieving the release member 1006 with the track-guide protrusion 1008 into the auto injector again, the index ring is rotated back to its initial rotational position R1.

The cassette housing 600 shown in FIG. 21 extends from a proximal end 602 to a distal end 604 and has an opening 612 at the distal end 604 through which the index ring 500, the needle safe cover parts 410, 440 and the syringe holder 300 are mounted. When the cassette 100 is assembled, the cassette housing 600 at least partly if not fully encloses the syringe holder 300, the syringe 200, 200d when positioned in the syringe holder 300, the needle safe cover parts 410, 440, and the index ring 500. The syringe holder 300 will normally be longitudinally and rotationally locked to the cassette housing 600. The cassette housing 600 has an inspection window 614 on at least one surface side allowing the user to inspect the syringe compartment 206, 206d containing the medicament when the syringe 200, 200d is mounted inside the cassette 100. The cassette 100 may be symmetrically constructed such that it may be mounted in an auto injector 1000 in different orientations. At the distal end 604 of the cassette housing 600, cassette securing tabs 616 are positioned. The cassette securing tabs 616 are flexible and are for securing the cassette 100 in the auto injector 1000.

The housing locking protrusion 606 on the inside of the cassette housing 600 interacts with the cover locking tab 430 of the first needle safe cover 410 as described above.

FIGS. 3A-L show the cassette 100 during mounting of the needle assembly 220 and medicament delivery in a partly cut-through perspective view, and FIGS. 4A-L show a cut-through the middle of the cassette 100. As shown in FIGS. 3A and 4A, the cassette 100 extends from a proximal end 102 to a distal end 104. In FIGS. 3A and 4A, the cassette 100 is in the delivery state before attaching the needle assembly 220. The proximal end 412 of the first needle safe cover 410 protrudes slightly outside the cassette housing 600. The first needle safe cover 410 is pushed proximally by the cover springs 470. However, further proximal movement of the first needle safe cover 410 into the fully extended position Pns 12L where the two needle safe cover parts 410, 440 are locked relative to each other is prevented by the release arms 480.

In FIGS. 3B and 4B, the needle assembly 220 rests against the inner support surface 428 and the proximally positioned surface plate 424. In FIGS. 3C and 4C, the needle assembly 220 and the first needle safe cover 410 has been pushed distally bringing the proximally positioned surface plate 424 of the first needle safe cover 410 in contact with the proximal end 442 of the second needle safe cover 440. Further distal movement of the two needle safe cover parts 410, 440 are prevented by the index ring 500 locking the second needle safe cover 440 longitudinally. The needle assembly 220 cannot be connected to the syringe holder 300 in this position, and the needle safe cover parts 410, 440 functions as a needle assembly blocking system.

In FIGS. 3D and 4D, the index ring 500 has been rotated from the first rotational position R1 as shown in FIGS. 3A-C, to the second rotational position R2 by the release member 1006 of the auto injector 1000 (not visible), which moves into the cassette 100 thereby rotating the index ring 500. Distal movement of the two needle safe cover parts 410, 440 are now possible when securing the needle assembly 220 to the syringe holder 300 as shown in FIGS. 3E and 4E. Upon securing the needle assembly 220 to the syringe holder 300, the release arms 480 are pushed from the initial position PraIN (as shown in FIGS. 3A and 4A-D) distally to the release position PraR. When the needle cap 228 is removed from the needle assembly 220, the cover springs 470 pushes the first needle safe cover 410 in the proximal direction causing the second needle safe cover 440 to follow suit. This is shown in FIGS. 3F and 4F. Depending on the strength of the cover springs 470, the movement pattern of the two needle safe covers 410, 440 may vary. If the two cover springs 470 has a high spring force than the needle safe cover spring system 1026 in the auto injector 1000 acting on the second needle safe cover 440, the two cover springs 470 will expand first and the second needle safe cover 440 initially stay behind. Alternatively, if the two cover springs 470 has a lower spring force than the needle safe cover spring system 1026 in the auto injector 1000 acting on the second needle safe cover 440, the two needle safe covers 410, 440 will initially move simultaneously until the end stop for the needle safe cover spring system 1026 after which, the cover springs 470 will expand for the respective end position of the first needle safe cover as shown in FIG. 3F.

When the safe cover locking protrusion 462 of the second needle safe cover 440 comes in contact with the index ring locking protrusion 506, the second needle safe cover 440 is prevented from moving further in the proximal direction as shown in FIGS. 3G and 4G.

However, since the release arms 480 are in the release position PraR, the first needle safe cover 410 is allowed to continue the proximal movement, which brings the first needle safe cover 410 into the extended position as shown in FIGS. 3H and 4H. In this fully extended position Pns12L, the first needle safe cover 410 and the second needle safe cover 440 are locked relative to each other forming one combined needle safe cover 408, which is allowed to move distally as one item. The distal movement of the combined needle safe cover 408 is shown in FIGS. 31 and 41 and is initiated when the user presses the cassette against an injector site for medicament delivery.

The auto injector may detect that the combined needle safe cover 408 has been pushed distally, e.g. by a switch being activated by the distal end 457 of the distally extending pin 456 coming into contact with needle safe cover pin 1024 activating the medicament delivery. Alternatively, the user may manually press a button on the auto injector 1000 and activate medicament delivery. During activation of the medicament delivery, the index ring 500 is rotated by the release member 1006, which moves further into the cassette 100 thereby rotating the index ring 500 even further. This is shown in FIGS. 3J and 4J.

A needle safe cover spring system 1026 in the auto injector 1000 pushes an injector needle safe cover pin 1024 proximally. The injector needle safe cover pin 1024 in turn is in contact with the distal end 457 of one of the distally extending pins 456 on the second needle safe cover 440. During medicament delivery, the injector needle safe cover pin 1024 moves proximally, whereby the needle safe cover spring system 1026 is biased. When the user removes the cassette from the injection site after end medicament delivery, the combined needle safe cover 408 is pushed proximally by the needle safe cover spring system 1026.

Since the index ring 500 is in the third rotation position R3, the combined needle safe cover 408 is now allowed to move into an extended position covering the needle 222. This is shown in FIGS. 3K and 4K. The first needle safe cover 410 is locked in position inside the cassette housing 600 as seen most clearly in FIG. 4K. When the release members 1006 retract distally into the auto injector 1000 again, the index ring 500 is rotated back to the first rotational position R1, where the index ring 500 prevents distal movement of the combined needle safe cover 408 as shown in FIGS. 3L and 4L. The cassette 100 is now locked in a position, where the needle 222 is protected and can be removed from the auto injector 1000. FIG. 5A shows an alternative example of the second needle safe cover 440′ where there is no difference between the first locking recess 460 and the surface 464. This means that in both the first rotational position R1 and the second rotational position R2 of the index ring 500, the needle assembly 220 can be secured to the syringe holder 300, since the second needle safe cover 440′ can be pushed distally. This is seen when comparing FIG. 5B and FIG. 5C Further rotational movement of the index ring 500 from the second rotational position R2 to a third rotational position R3 moves the index ring locking protrusion 506 completely out of contact with the safe cover locking protrusion 462 and first locking recess 460. Normally, the auto injector 1000 rotates the index ring 500 to the third rotational position R3 when the medicament delivery is initiated by proximal movement of the plunger rod 1018 in the auto injector. The medicament delivery position is shown in FIG. 5D, and the final locked position after medicament delivery is shown in FIG. 5E.

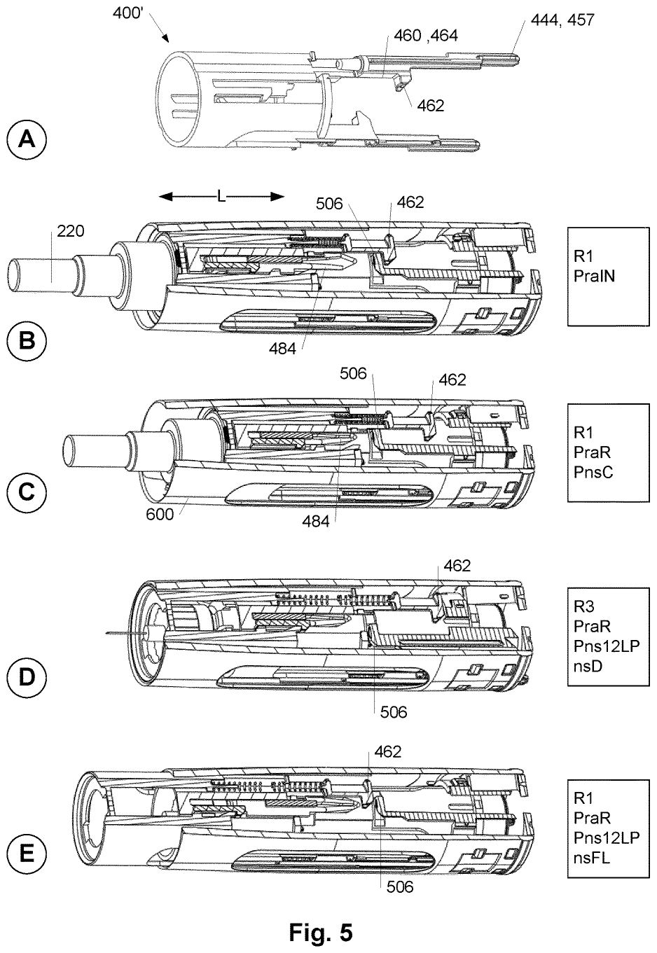

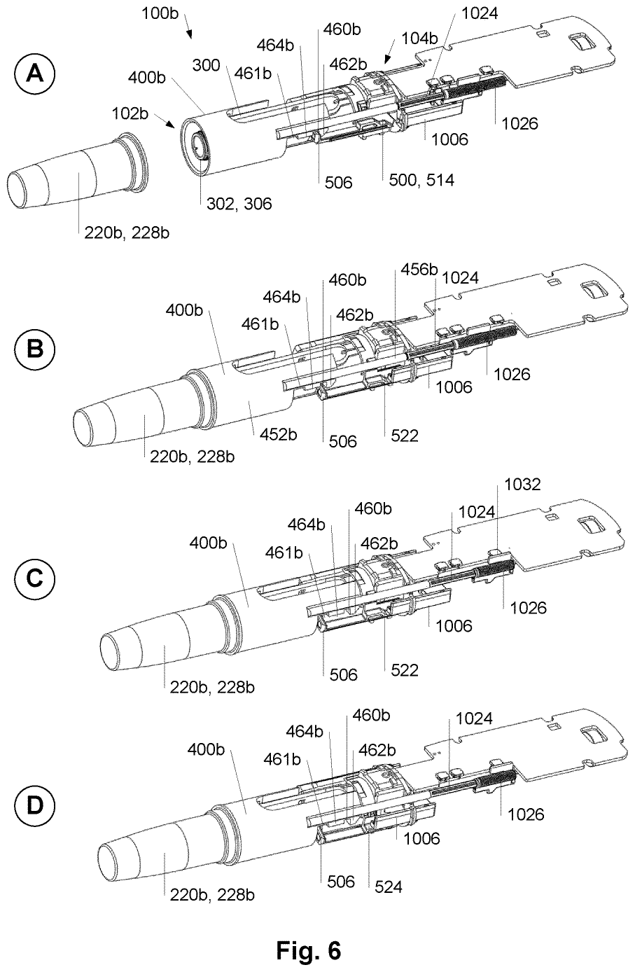

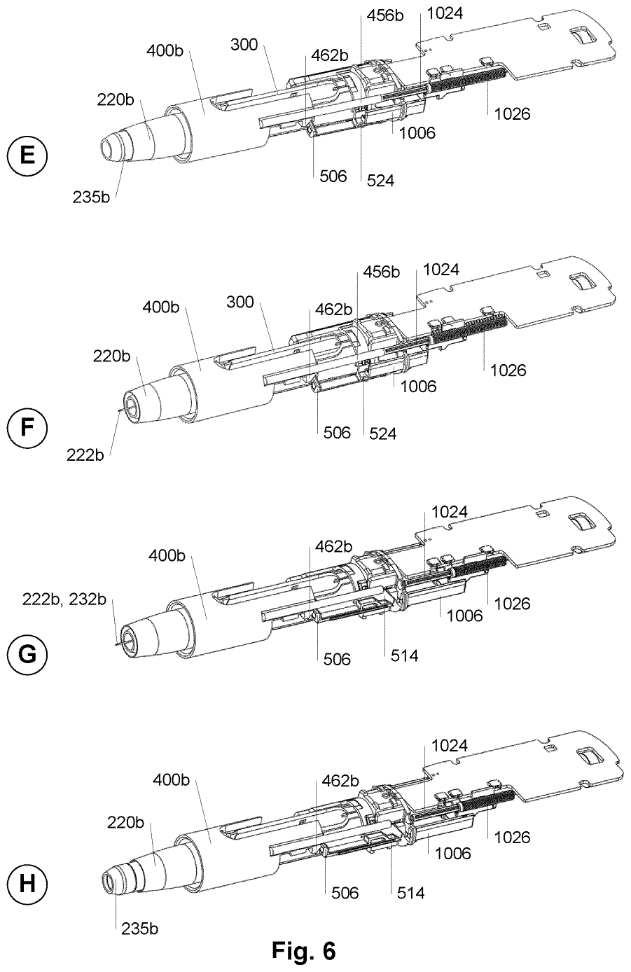

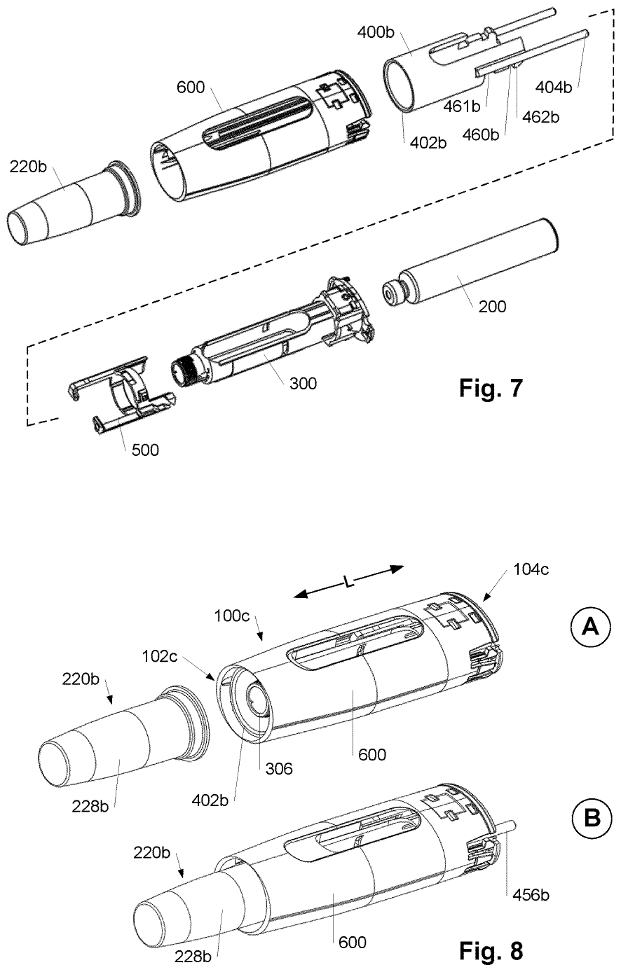

FIGS. 6A-H show a second cassette 100b during mounting of a needle assembly 220b and during medicament delivery. FIG. 7 shows an exploded view of the different parts in the second example of the cassette 100b and a second example of the needle assembly 220b. The similar parts are marked with “b”, but the same reference numbers are used for similar parts. The cassette 100b extends from a proximal end 102b to a distal end 104b as shown in FIG. 6A.

Compared to the needle assembly 220 shown and described in FIGS. 1, 2A-1, 3A-L and 4A-L, the needle assembly 220b configured for being connected to the second cassette 100b includes a needle assembly shield 235b, which is deflectable in the distal direction for exposure of the needle 222b. A needle assembly shield spring (not shown) pushes the needle assembly shield 235b in the proximal direction after use for protecting the needle 222b. Other parts of the needle assembly 220b also includes a needle hub 224b, a needle assembly adaptor 226b, and a needle cap 228b. The needle cap 228b is removable from the needle assembly 200b after securing the needle assembly 220b to the syringe holder 300 by securing the needle assembly adaptor 226b to the needle assembly locking part 306 of the syringe holder 300.

Compared to the cassette 100 shown and described in FIGS. 1, 2A-1, 3A-L and 4A-L, the cassette 100b shown in FIG. 7 in an exploded view is absent of a telescopically extendable assembly, but instead comprises a one-part needle mounting blocking system 400b. The needle mounting blocking system 400b comprises similar parts as the second needle safe cover 440 as shown and described in FIGS. 1, 2A-1, 3A-L and 4A-L. The needle mounting blocking system 400b extends from a proximal end 402b to a distal end 404b.

The needle mounting blocking system 400b has a tubular part 452b. Extending distally from the tubular part 452b are distally extending pins 456b. The needle mounting blocking system 400b is normally symmetrically with a distally extending pin 456b on both sides of the needle mounting blocking system 400b.

The distal end 457b of the distally extending pin 456b interacts with the auto injector 1000, possibly by activating a sensor informing the auto injector 1000 that a cassette 100b has been secured in the auto injector 1000. Alternatively, a cassette insertion detection sensor system may be positioned at a different location in the auto injector 1000.

The distally extending pin 456b comprises a first locking recess 460b and a blocking system locking protrusion 462b. The blocking system locking protrusion 462b is positioned distally of the first locking recess 460b. The first locking recess 460b and blocking system locking protrusion 462b interacts and couples with the index ring 500. The index ring 500 (see FIG. 2H) comprises an index ring locking protrusion 506, which is secured inside the first locking recess 460b prior to securing the cassette 100b in the auto injector 1000. When the index ring locking protrusion 506 is positioned fully in the first locking recess 460b, the needle mounting blocking system 400b is longitudinally locked to the index ring 500. Since the index ring 500 is normally longitudinally locked to the syringe holder 300, the needle mounting blocking system 400b is longitudinally locked to the syringe holder 300 when the index ring locking protrusion 506 is positioned fully in the first locking recess 460b.

The index ring 500 is rotatable relative to the needle mounting blocking system 400b. The rotation of the index ring 500 is controlled by movement of one or more release members 1006 in the auto injector 1000 into/out of the cassette 100b. When the index ring 500 is rotate from an initial first rotational position R1 to a second rotation position R2, the index ring locking protrusion 506 moves to a position where it is no longer prevents the needle mounting blocking system 400b from moving distally. This can be seen by comparing FIGS. 6A-C. In the second rotational position R2, the needle mounting blocking system 400b is prevented from moving proximally by the blocking system locking protrusion 462b. The blocking system locking protrusion 462b extends further radially than the surface 464b of the distally extending pin 456b found on the proximal side of the first locking recess 460b. In some examples, there is no difference between the first locking recess 460b and the surface 464b. In the second rotational position R2, the needle assembly 220b can be secured to the syringe holder 300, since the needle mounting blocking system 400b can be pushed distally. In FIG. 6C, the needle mounting blocking system 400b has been pushed distally and the needle assembly 220b mounted.

FIGS. 6A-H show the second cassette during mounting of the needle assembly 220b and medicament delivery and the interacting with the auto injector 1000 during the process. The rotation of the index ring 500 by the release member 1006 in the injector 1000 can be seen when comparing FIGS. 6A-H. In FIG. 6A, the cassette 100b has just been mounted in the auto injector 1000. In FIG. 6B, the auto injector 1000 has detected the presence of the cassette 100b and has locked the cassette 100b to the auto injector 1000 and moved the release member 1006 proximally to rotate the index ring 500 to the second rotational position R2. When pushing the needle assembly 220b distally securing it to the cassette 100b as shown in FIG. 6C, a switch 1032 is activated. This result in the release member 1006 moving further proximally to rotate the index ring 500 to the third rotational position R3 as shown in FIG. 6D. When the needle cap 228b is removed, the needle mounting blocking system 400b moves proximally as shown in FIG. 6E. For delivery of medicament, the auto injector 1000 and cassette 100b system is pressed against the patient's skin and a needle assembly shield 235b of the needle assembly 220b is pushed distally. This is seen in FIG. 6F. After medicament delivery, the release member 1006 in the injector 1000 retracts distally thereby rotating the index ring 500 to the first rotational position R1 as shown in FIG. 6G. When the cassette 100b is removed from the patient's skin, a needle assembly shield spring inside the needle assembly 220b forces the needle assembly shield 235b of the needle assembly 220b proximately to cover the needle 222b as shown in FIG. 6H.

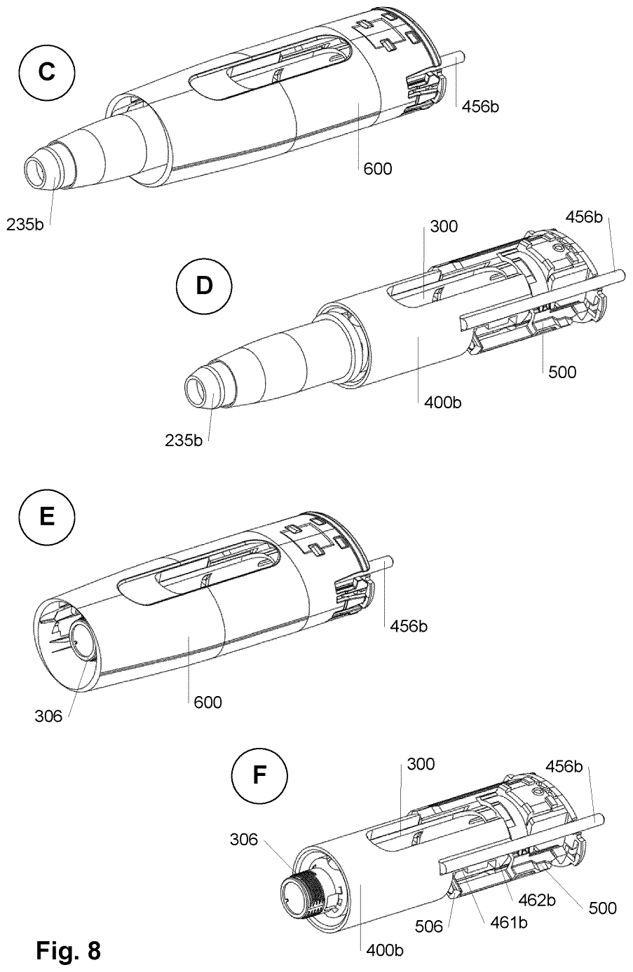

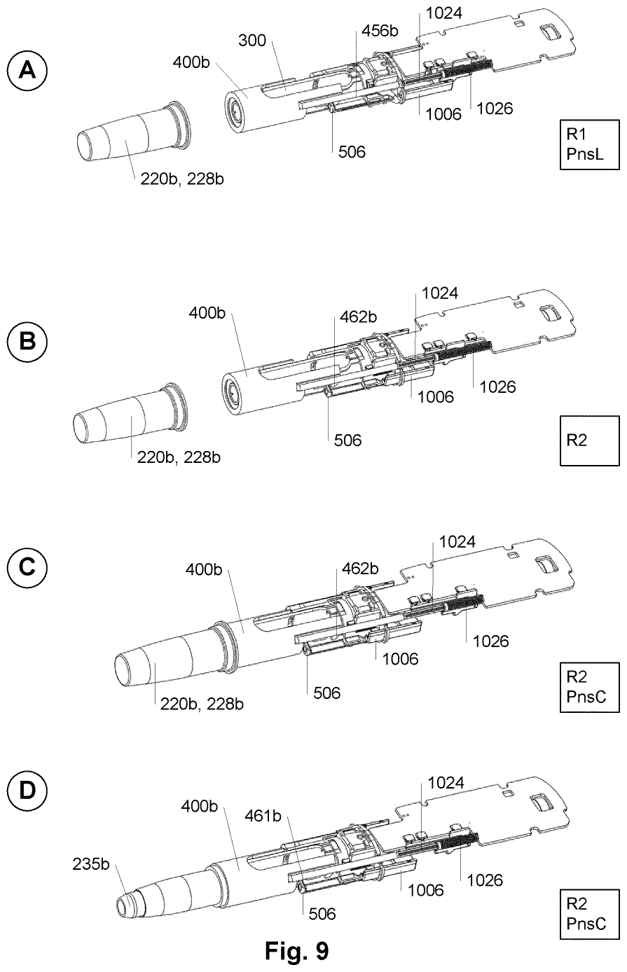

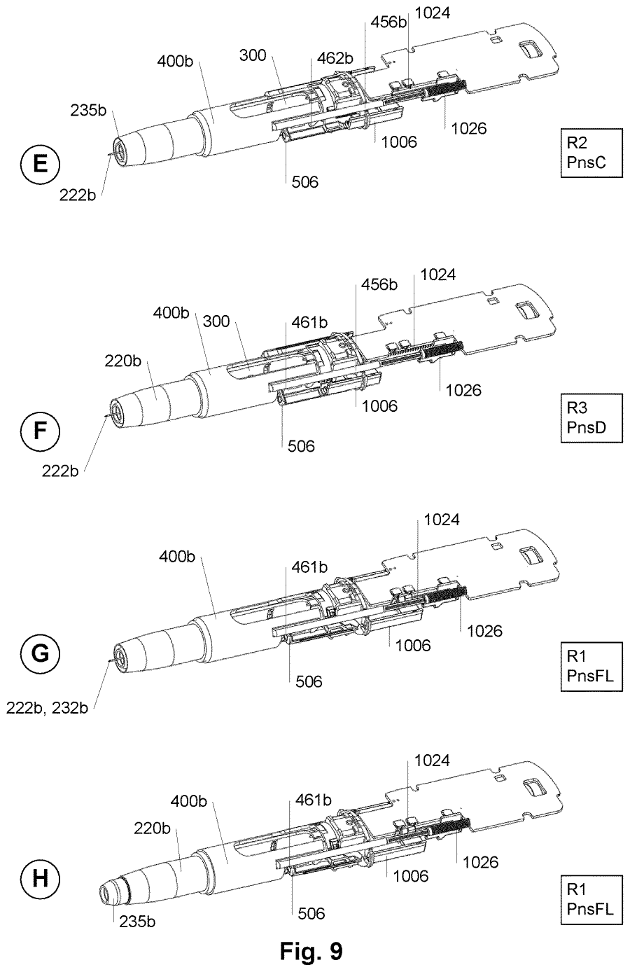

FIGS. 8A-F show a third cassette 100c extending from a proximal end 102c to a distal end 104c before connecting to a needle assembly 220b, during mounting of the needle assembly 220b and during medicament delivery. The similar parts are marked with “c”, but the same reference numbers are used for similar parts as compared to the previously described first and second examples of the cassette 100, 100b. FIGS. 9A-H show the third cassette 100c during mounting of the second example of the needle assembly 220b and during medicament delivery in similar views as in FIGS. 6A-H.

In FIG. 8A, the cassette 100c is in a pre-use position, where the needle mounting blocking system 400b is in the unlocked initial position Pns1IN. In FIGS. 8B-D, the cassette 100c is in a position where the needle assembly 220b has been secured to the cassette 100c and the needle mounting blocking system 400b is in the needle assembly connecting position PnsC. In FIGS. 8E-F, the cassette 100c is an after-use position, where the needle mounting blocking system 400b is in the final locked position PnsFL. Apart from the cassette housing 600 being removed from the cassette 100c in FIG. 8D and FIG. 8F, FIG. 8D and FIG. 8F are identical to FIG. 8C and FIG. 8E, respectively.

In FIG. 9A, the cassette 100c has been inserted into the auto injector 1000 but not yet secured in it. The index ring 500 has therefore not yet rotated from the initial first rotational position R1 to a second rotation position R2. Mounting of the needle assembly 220b is therefore not yet possible. In FIG. 9B, the cassette 100c has been locked in the auto injector 1000 and the index ring 500 rotated to the first rotational position R1 where the index ring locking protrusion 506 has been moved to a position where it is no longer prevents the needle mounting blocking system 400b from moving distally. In the second rotational position R2, the needle assembly 220b can be secured to the syringe holder 300, since the needle mounting blocking system 400b can be pushed distally. In FIG. 9C, the needle mounting blocking system 400b has been pushed distally and the needle assembly 220b mounted. In FIG. 9D, the needle cap 228b is removed. For delivery of medicament, the auto injector 1000 and cassette 100c system is pressed against the patient's skin and a needle assembly shield 235b of the needle assembly 220b is pushed distally. This is seen in FIG. 9E. The plunger rod 1018 moved proximately to push medicament out of the syringe 200, 200d. This is seen in FIG. 9F. After medicament delivery, the release member 1006 in the injector 1000 retracts distally thereby rotating the index ring 500 to the first rotational position R1 as shown in FIG. 9G. When the cassette 100c is removed from the patient's skin, a needle assembly shield spring inside the needle assembly 220b forces the needle assembly shield 235b of the needle assembly 220b proximately to cover the needle 222b as shown in FIG. 9H. When the index ring 500 has rotated to the first rotational position R1, the cassette 100c is in a final locked position PnsFL from where the needle mounting blocking system 400b is prevented from moving distally or proximally. This is obtained by the index ring locking protrusion 506 now fitting into a second locking recess 461b of the distally extending pin 456b. When the cassette 100c has been used for medicament delivery, the needle mounting blocking system 400b is thus in a final locked position PnsFL, which is distal of the locked position PnsL. The final locked position PnsFL is therefore the same as the needle assembly connecting position PnsC and the medicament delivery position PnsD.

The index ring may differ from that shown in FIGS. 9A-H in that it does not rotate to a third rotational position R3. The index ring 500 used in the cassette 100c may lack an equivalent to the third track section 524 of the first example of the index ring 500. During medicament delivery, the index ring 500 may be at the second rotation position R2. The needle safe cover spring system 1026 in the auto injector 1000 need not push the needle mounting blocking system 400b proximately into an extended position, if the distally extending pins 456b are not extending far enough in the distal direction for the needle safe cover spring system 1026 to exert a proximal-pushing force on the distally extending pins 456b. After medicament delivery when the auto injector 1000 retracts the one or more release members 1006, the index ring 500 rotates from the second rotational position R2 to the first rotational position R1.

FIGS. 10A-C shows the dual chamber syringe 200d with a first sub-compartment 214, and a second sub-compartment 216 as described previously.

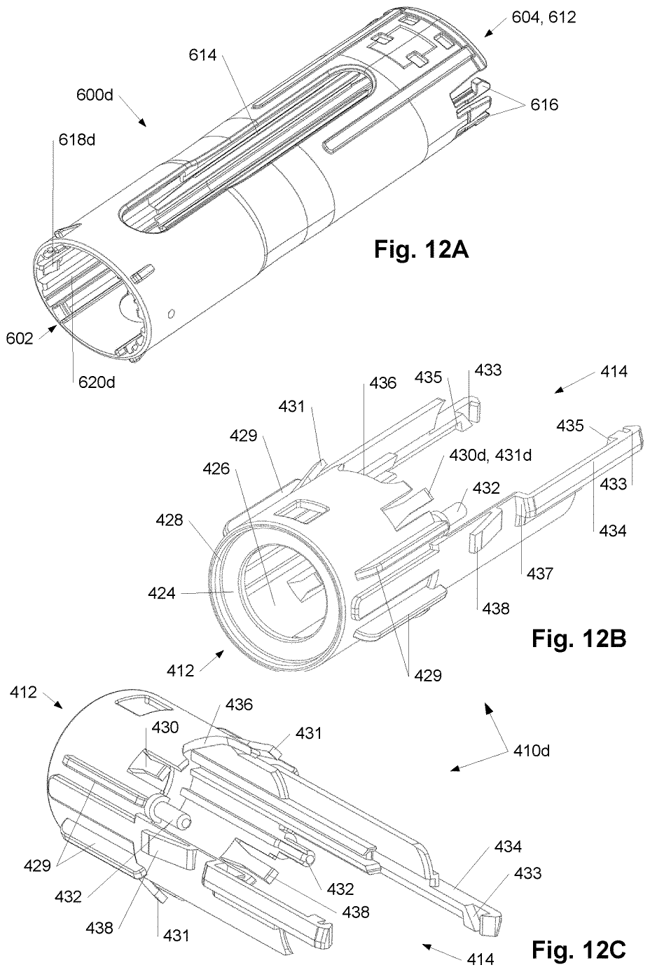

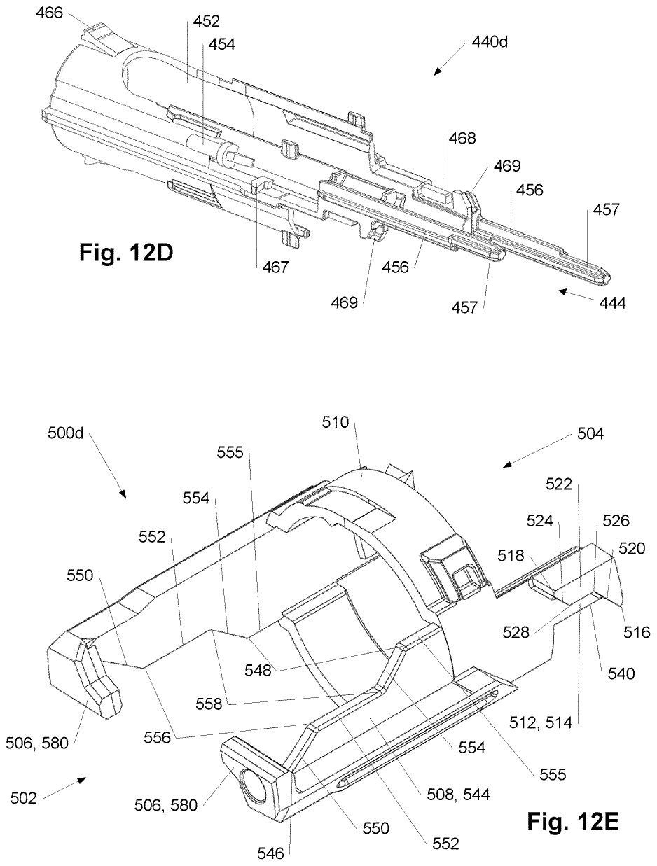

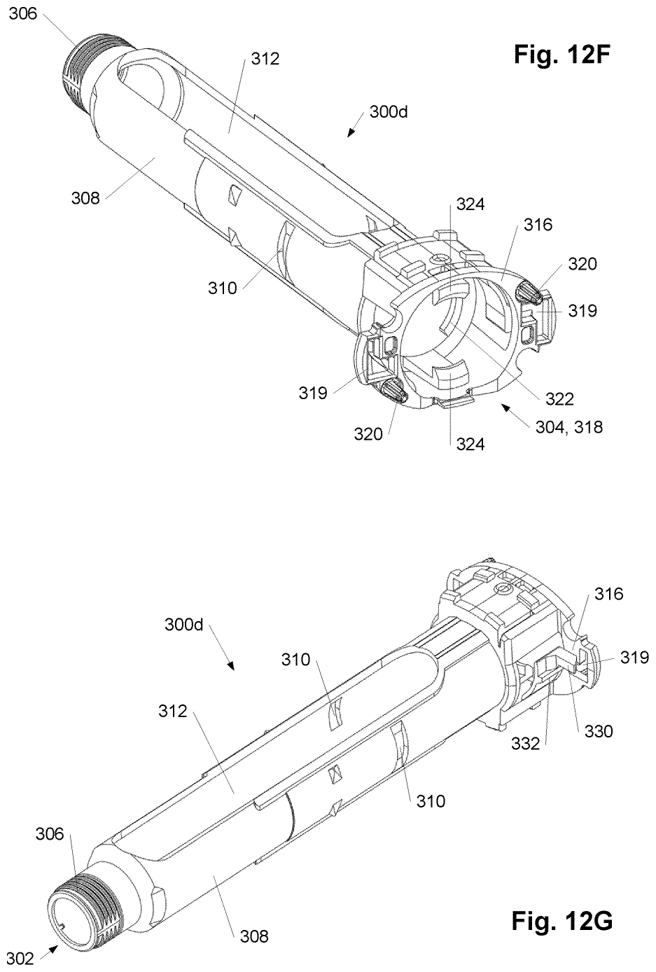

FIG. 11 shows a fourth cassette 100d in an exploded view. The fourth cassette 100d comprises a syringe holder 300d, a first needle safe cover 410d, a second needle safe cover 440d, a cover spring 470, an index ring 500d, and a cassette housing 600d. Apart from the cover spring 470, the other cassette parts 410d, 440d, 500d, 600d are slightly alternative examples of the previously discussed parts in the first example of the cassette 100. The similar parts are marked with “d”, but the same reference numbers are used for similar parts as compared to the previously described first, second and third examples of the cassette 100, 100b, 100c. Comparing to the first example of the cassette 100, the fourth example of the cassette 100d lacks separate release arms. Instead, the release arms 580 in the fourth example of the cassette 100d are positioned as a part of the index ring 500d as shown in FIG. 12E.

FIG. 12A show the cassette housing 600d. In addition to parts already described for the first version of the cassette housing 600, the cassette housing 600d shown in FIG. 12A comprises a first needle safe cover stop surface 618, which prevents the first needle safe cover 410d from being pushed proximally out of the cassette housing 600d in the final locked position by abutting a distal end cover locking tab 431 on the first needle safe cover 410d. The cassette housing 600d further comprises at least one first needle safe cover channel 620 in which a cassette housing guide tab 429 travels. The channel 620 and guide tab 429 pair rotationally locks the cassette housing 600d and the first needle safe cover 410d.

FIGS. 12B-C show the first needle safe cover 410d from two different directions. In addition to the cassette housing stop surface 438 and the cassette housing guide tab 429 described above, and the parts already described for the first version of the first needle safe cover 410, the first needle safe cover 410d shown in FIGS. 12B-C comprises a cover locking tab 430 with the distal end cover locking tab 431d preventing the first needle safe cover 410d from being pushed proximally out of the cassette housing 600d in the final locked position. The first needle safe cover 410d further comprises a distal first needle safe cover locking surface 436, and a proximal first needle safe cover locking surface 437. In the Pns 12L position, the distal first needle safe cover locking surface 436 abuts a proximal second needle safe cover locking tab 466 on the second needle safe cover 440d preventing distal movement of the first needle safe cover 410d relative to the second needle safe cover 440d. Likewise, in the Pns12L position, the first needle safe cover interface 435 abuts a distal second needle safe cover locking tab 467 on the second needle safe cover 440d preventing proximal movement of the first needle safe cover 410d relative to the second needle safe cover 440d. In this way, the two needle safe cover parts 410d, 440d are locked relative to each other in the Pns12L position.

The first needle safe cover 410d also comprises a first needle safe cover arm 434 with a first index ring engaging finger 433 and a first needle safe cover interface 435. When the index ring 500d is in the first rotational position R1, the first index ring engaging finger 433 on the first safe cover 410d abut at least one of the one or more release arms 580 on the index ring 500d preventing proximal movement of the first needle safe cover 410d relative to the index ring 500d. When the first needle safe cover 410d and the second needle safe cover 440d are in the extended locked position, the first needle safe cover interface 435 abuts against distal second needle safe cover locking tab 467 prevention further proximal movement of the first needle safe cover 410d relative to the second needle safe cover 440d.

FIG. 12D show the second needle safe cover 440d. In addition to the proximal second needle safe cover locking tab 466, and the distal second needle safe cover locking tab 467 described above, and the parts already described for the first version of the second needle safe cover 440, the second needle safe cover 440d comprises a second index ring engaging finger 469 and a second needle safe cover interface 468. The second needle safe cover interface 468 keeps the two needle safe cover parts 410d, 440d together when assembled with the index ring 500d. The second needle safe cover interface 468 abuts the release arm 580 prior to positioning of the cassette 100d in the auto injector 1000. The cover springs 470 positioned around the proximally extending pin 454 on the second needle safe cover 440d and the distally extending pin 432 on the first needle safe cover 410d, will try to push the needle safe cover parts 410d, 440d away from each other. However, prior to the relative release of the needle safe cover parts 410d, 440d by the release arms 580 on the index ring 500d, the release arms 580 lock the needle safe cover parts 410d, 440d together in the unlocked initial position Pns1IN.

FIGS. 12F-G show the syringe holder 300d from two different directions. In addition to the index ring release arm 330 with the protruding surface 332 and the parts already described for the first version of the syringe holder 300, the syringe holder 300d comprises cassette detection tabs 320 (also present in the first version of the syringe holder 300 as shown in FIG. 2C) abutting a cassette detection pin 1080 inside the auto injector 1000 for cassette detection. The syringe holder 300d shown in FIGS. 12F-G further comprises syringe support surfaces 322 and syringe securing hook 324 for supporting and securing the syringe 200, 200d in the syringe holder 300d. These parts may be omitted.

FIG. 12E shows the index ring 500d. In addition to the parts already described for the first version of the index ring 500, the index ring 500d shown in FIG. 12E comprises a second track 544 positioned on the index ring arm 508. The second track 544 comprises a first secondary track section 550, a second secondary track section 552, a third secondary track section 554, and a fourth secondary track section 555. The first secondary track section 550 extends from the first secondary corner 546 in a first angled direction towards a first secondary middle point 556. The second secondary track section 552 extends from the first secondary middle point 556 to a second secondary middle point 558 along the longitudinal direction of the cassette 100d in a second angled direction. The third secondary track section 554 extends from the second secondary middle point 558 in a third angled direction towards the second secondary corner 548. The fourth secondary track section 555 extends from the second secondary corner 548 and distally. The first and/or third angled directions are normally sloping directions, with an angle between 20 and 70 degrees, such as between 30 and 60 degrees, such as between 35 and 55 degrees, such as between 40 and 50 degrees, such as approximately 45 degrees relative to the longitudinal direction of the cassette. The second secondary track section 522 is normally a plateau track section with a second angled direction between −20 and 20 degrees, such as between −10 and 10 degrees, such as approximately 0 degrees relative to the longitudinal direction of the cassette. The fourth secondary track section 555 is likewise normally a plateau track section with a fourth angled direction between −20 and 20 degrees, such as between −10 and 10 degrees, such as approximately 0 degrees relative to the longitudinal direction of the cassette.

The index ring 500d is locked relative to the needle safe covers 410d, 440d prior to inserting the cassette 100d in the auto injector 1000. This is due to a blocking surface 540 on the index ring 500d abutting a protruding surface 332 on an index ring release tab 330 on the syringe holder 300d. When the cassette 100d is positioned in the auto injector 1000, the one or more release members 1006 in the auto injector 1000 travels proximally into the cassette 100d thereby coming into contact with the index ring release tab 330, which causes the index ring release tab 330 to flex radially inwardly. By doing so, the blocking surface 540 on the index ring 500d no longer abuts the protruding surface 332. This makes the index ring 500d rotatable from the first rotational position R1 to the second rotational position R2.

The rotation of the index ring 500d from the first rotational position R1 towards the second rotational position R2 is controlled by the user attaching a needle assembly 220 to the needle assembly locking part 306 on the syringe holder 300d by pushing the needle safe cover parts 410d, 440d distally. The second index ring engaging finger 469 on the second needle safe cover 440d abuts the first secondary track 550 of the second track 544 therefore forcing the index ring 500d to rotate towards the second rotational position R2 as the user pushes the needle safe cover parts 410d, 440d distally.

The rotation of the index ring 500d results in the release arms 580 being released from the first index ring engaging finger 433. This allows for the cover springs 470 to move the first needle safe cover 410d proximately relative to the second needle safe cover 440d after needle assembly 220 mounting. The cover springs 470 proximately movement of the first needle safe cover 410d relative to the second needle safe cover 440, pushes the needle safe covers 410d, 440d into a locked cover position where further relative longitudinal movement between the first needle safe cover 410d and the second needle safe cover 440d is prevented. The proximal locking is obtained by a first locking pair constituting the proximal first needle safe cover interface 435 and the distal second needle safe cover locking tab 467, as described above. The distal locking is obtained by a second locking pair, constituting the distal first needle safe cover locking surface 436 and the proximal second needle safe cover locking tab 466, as described above.

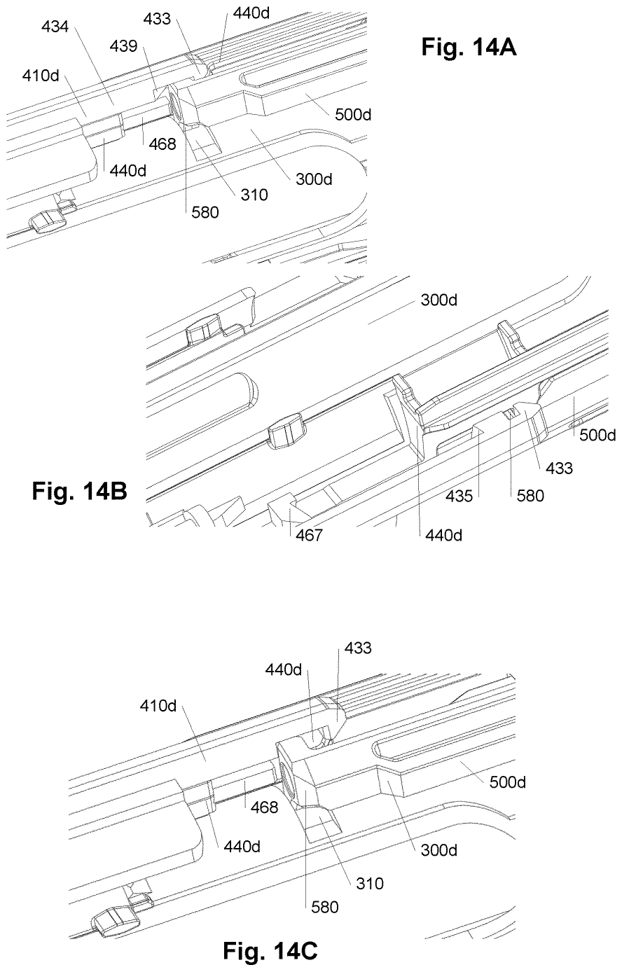

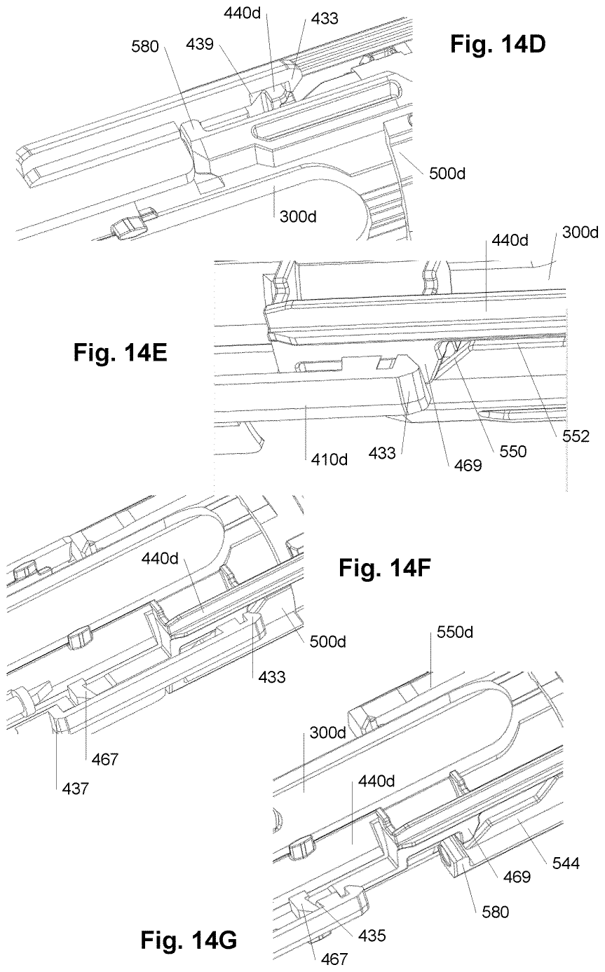

In the second rotational position R2 of the index ring 500d, the second index ring engaging finger 469 on the second needle safe cover 440d abuts the release arm 580 preventing further proximal movement of the needle safe covers 410d, 440d. This is seen in FIG. 14G.

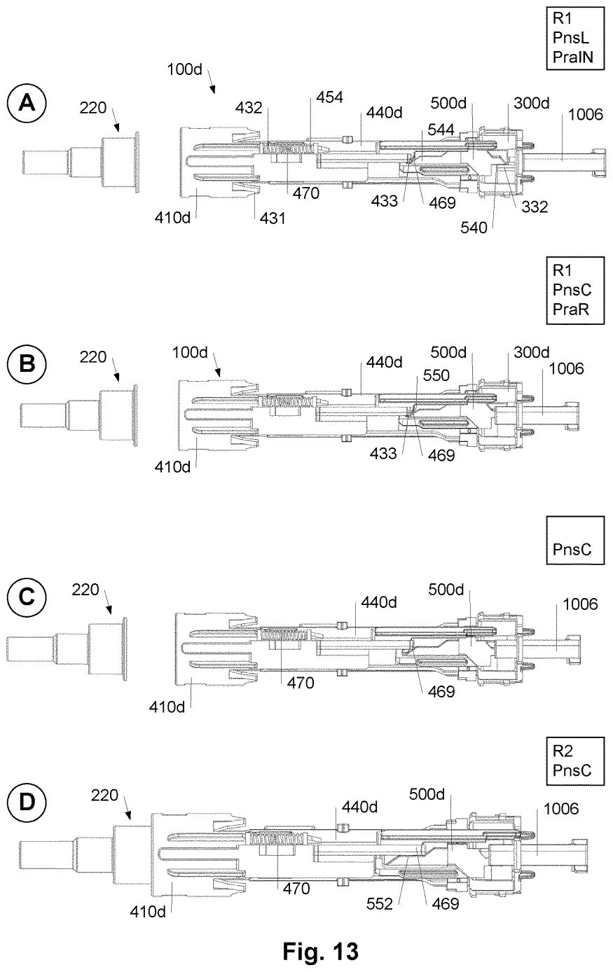

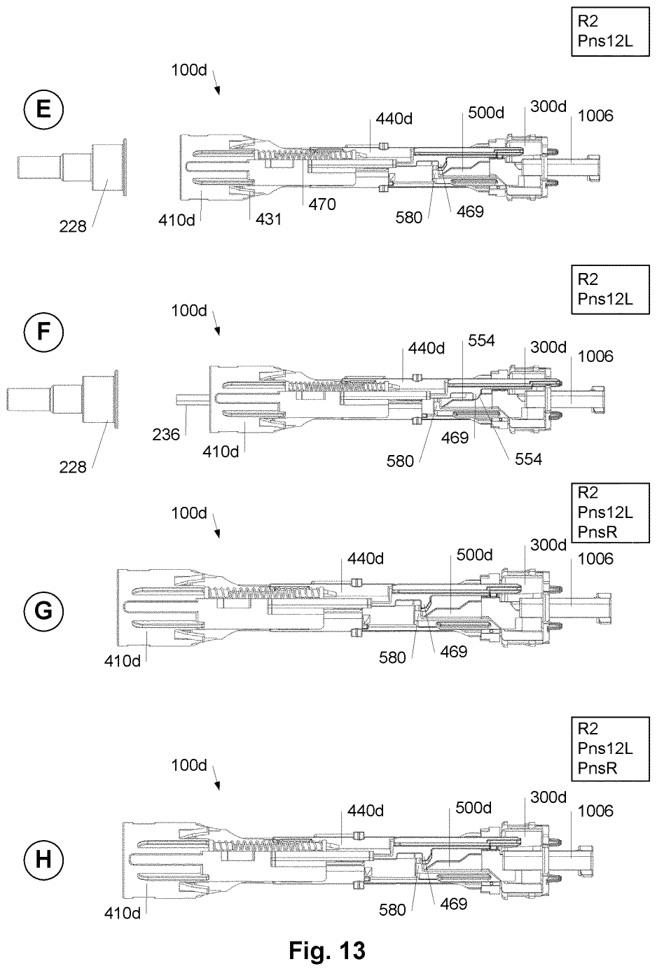

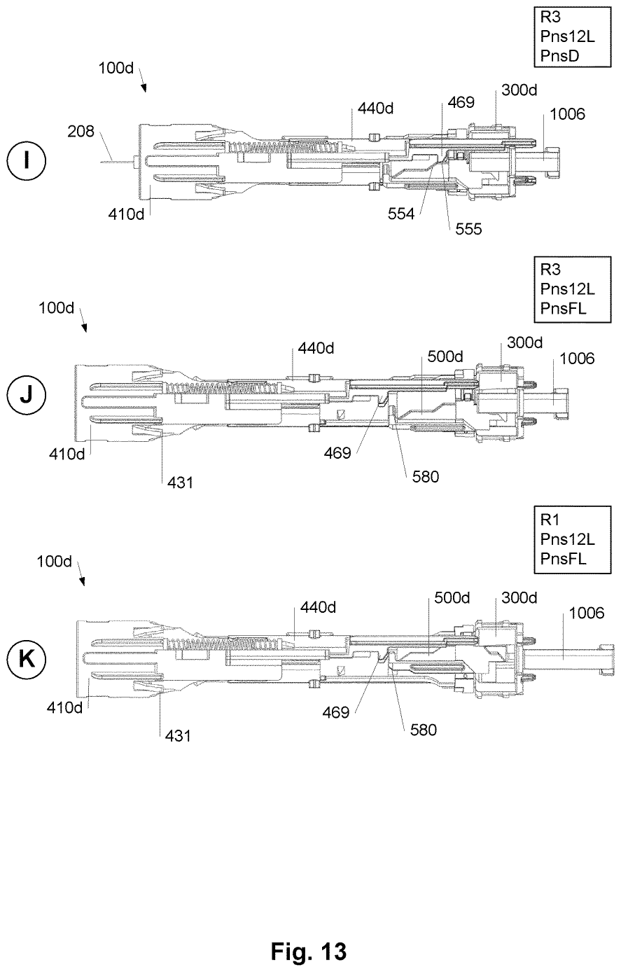

The index ring 500d is rotatable from the second rotational position R2 to the third rotation position R3 after proximal movement of the one or more release members 1006 of the auto injector from the first release member position inside the cassette 100d to a second release member position inside the cassette 100d. The index ring 500d can be rotated from the second rotational position R2 to the third second rotational position R3 by movement of the first needle safe cover 410d distally after the proximal movement of the one or more release members 1006 of the auto injector 1000. This is done when the user presses the first needle safe cover 410d against the injection site for needle insertion. See FIGS. 13H-I for more details.

When the index ring 500d is rotated from the second rotational position R2 to the third second rotational position R3 by movement of the first needle safe cover 410d distally for needle insertion and medicament delivery, the second index ring engaging finger 469 no longer abuts the release arms 580 in the proximal direction. This allows for proximal movement of the needle safe covers 410d, 440d relative to the index ring 500d to a final locked position PnsFL after end medicament delivery. Thus, the second index ring engaging finger 469 is allowed to move proximally of the release arms 580 to the final locked position PnsFL as shown in FIGS. 13J-K.

FIGS. 13A-K show a sequence similar to that shown in FIGS. 3A-L with the forth cassette during mounting of the needle assembly and subsequent medicament delivery in a cut-through view. In FIG. 13A, the cassette 100d is yet to be inserted into the auto injector and the needle assembly 220 has not yet been attached to the cassette 100d. In FIG. 13B, the cassette 100d has been positioned inside the auto injector and the release member 1006 has moved proximally for locking the cassette 100d inside the auto injector. The index ring 500d has not been rotated yet, but is now rotatable from the first rotational position R1 to the second rotational position R2 by user mounting of the needle assembly 220. In FIG. 13C, the first needle safe cover 410d has been moved distally relative to the second needle safe cover 440d. This rotates the index ring 500d slightly (see FIG. 14C) and compresses the cover spring 470 further compared to what is shown in FIG. 13B.

In FIG. 13D, the needle assembly 220 has been secured to the syringe holder 300d and the index ring 500d rotated to the second rotational position R2 by the second index ring engaging finger 469 traveling along the first and the second secondary tracks 550, 552 of the second track 544 (see closeups in FIGS. 14D-F). The first needle safe cover 410d is unlocked relative to the second needle safe cover 440d in this position. In FIG. 13E, the needle cap 228 has been removed, whereby the first needle safe cover 410d pushed to a proximally extending position covering the needle 208 by the spring force of the cover spring 470. The release arms 580 prevent movement to the outermost proximal position of the needle safe covers 410d, 440d by the abutment with the second index ring engaging finger 469. However, in this position, the needle safe covers 410d, 440d are relatively locked. In FIG. 13F, the needle safe covers 410d, 440d are pushed distally for removal of a rigid needles shield 236. After removal of the rigid needles shield 236, the needle safe covers 410d, 440d again moves to the position of FIG. 13E due to the needle safe cover spring system 1026 of the auto injector.

FIG. 13H illustrates the movement of the plunger rod 1018 and the release member 1006 for mixing of medicament in the two sub-compartments 214, 216 when a dual chamber syringe 200d is used. If a single chamber syringe 200 is used, this step can be omitted.

In FIG. 131, the needle 208 is inserted into the injection site by pushing the first needle safe cover 410d against the injection site and medicament is delivered. Pushing the first needle safe cover 410d against the injection site also rotates the index ring 500d from the second rotational position R2 to the third rotational position R3 by the second index ring engaging finger 469 traveling along the third secondary track section 554. After traveling along the third secondary track section 554, the second index ring engaging finger 469 travels along the fourth secondary track section 555 as shown in FIG. 131.

The rotation of the index ring 500d to the third rotational position R3 allows the second index ring engaging finger 469 to pass by the release arm 580 in the proximal direction after removal of the device from the injection site. This is shown in FIG. 13J. The distal end of the cover locking tab 431 now locks the first needle safe cover 410d in the extended proximal position, i.e. the final locked position PnsFL. By distal movement of the release member 1006 as shown in FIG. 13K, the index ring 500d is rotated back to the first rotational position R1 and the cassette 100d is now removed from the auto injector.

FIGS. 14A-G show a close-up of the interaction between the first needle safe cover 410d, the second needle safe cover 440d, the index ring 500d and the syringe holder 300d during the process of mounting a needle assembly 220 to the syringe holder 300d. In FIGS. 14A-B (being illustrations shown from different directions), the cassette 100d is in the original position prior to mounting of the cassette 100d in the auto injector. The release arm 580 is positioned in the index ring receiving opening 310 and between the first index ring engaging finger 433 and a sloping surface 439 on the first needle safe cover arm 434. The second needle safe cover interface 468 on the second needle safe cover 440d is also visible in FIG. 14A being positioned against the release arm 580. The first needle safe cover interface 435 and the distal second needle safe cover locking tab 467 can be seen in FIG. 14B.

When cassette 100d has been locked in the auto injector 1000 and the index ring 500d rotationally released from the syringe holder 300d by the proximal movement of the release member 1006 in the auto injector as shown in FIG. 13B, the first needle safe cover 410d can be moved distally relative to the second needle safe cover 440d. This rotates the index ring 500d slightly due to the sloping surface 439 on the first needle safe cover arm 434 abutting against the release arm 580 on the index ring 500d. This moves the system to the position as shown in FIG. 14C being a close-up of the position shown in FIG. 13C. The needle assembly 220 can now be attached.

In FIGS. 14D-F (being illustrations shown from different directions), the needles assembly is attached. When doing so, the first needle safe cover 410d and the second needle safe cover 440d are moved further distally. This rotates the index ring 500d to the second rotational position R2 due to the second index ring engaging finger 469 traveling in the second track 544 against the first and second secondary tracks 550, 552 of the second track 544. This moves the system to the position shown in FIG. 13D. The needle assembly 220 is now be attached.

When the second index ring engaging finger 469 travels in the secondary track 552 of the second track 544 the index ring 500d it rotated to the second rotational position R2. The index ring 500d rotation moves the release arm 580 out of engagement with the first index ring engaging finger 433. When the needle cap 228 is removed, the first needle safe cover 410d moves proximally relatively to the second needle safe cover 440d due to the cover spring 470 pushing the two cover parts away from each other. The two needle safe covers 410d, 440d are now locked in the extended position as shown in FIG. 13E. The proximal locking of the two needle safe covers 410d, 440d is obtained by the first needle safe cover interface 435 and the distal second needle safe cover locking tab 467.

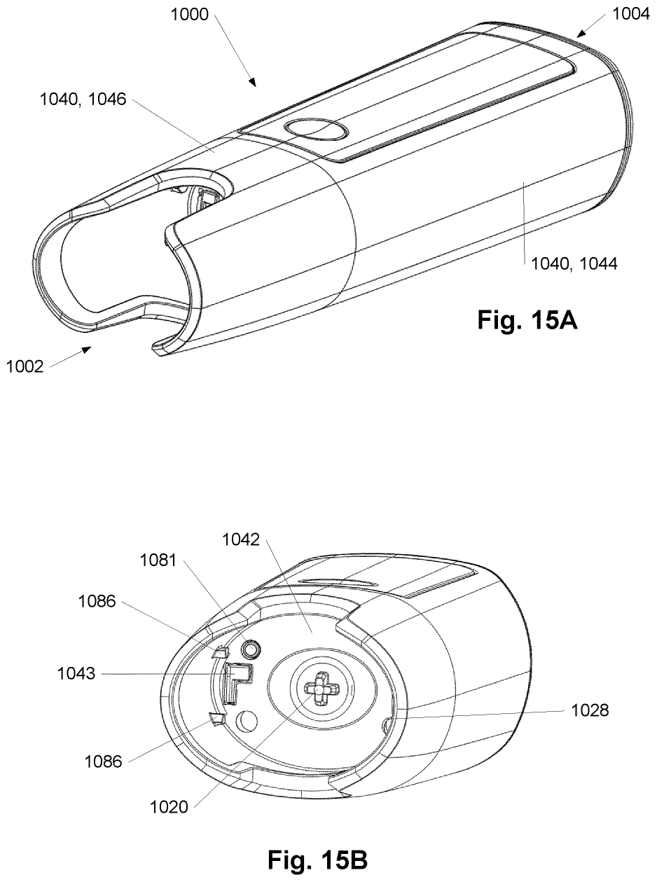

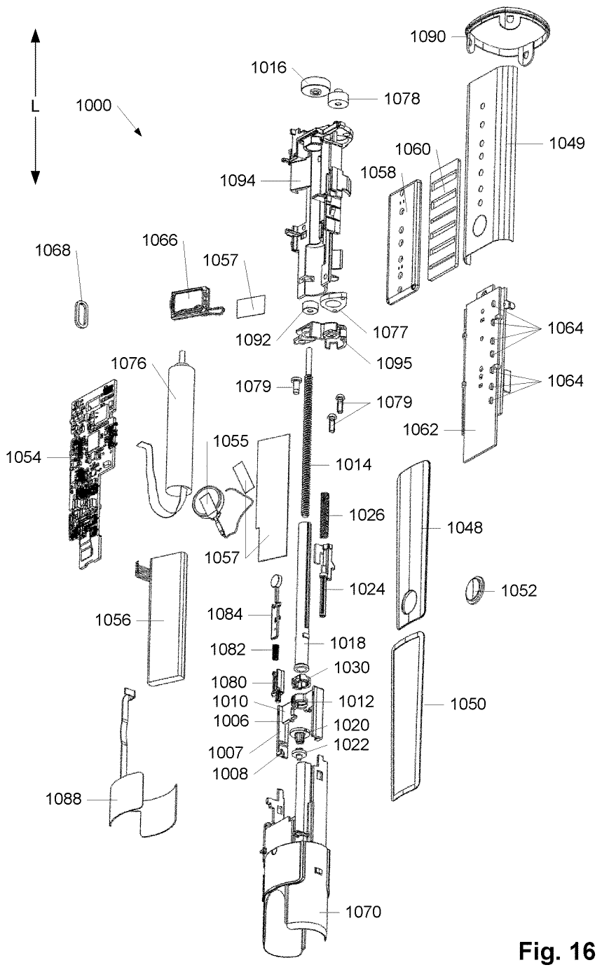

FIGS. 15A-B show the auto injector 1000 in an assembled view, and FIG. 16 shows an exploded view of the auto injector 1000 configured for receiving a cassette 100, 100b, 100c, 100d. All examples of the cassette 100, 100b, 100c, 100d described above can be received in the auto injector 1000. Different cassette versions may also in received in the auto injector 1000. The auto injector 1000 is extending from a proximal end 1002 to a distal end 1004 along a longitudinal axis L, and comprises an injector housing 1040 and a multiple of internal injector parts positioned inside the injector housing 1040.

The cassette 100, 100b, 100c, 100d is removable received in the auto injector 1000 along the longitudinal direction L. Normally, the cassette 100, 100b, 100c, 100d will be received at the proximal end 1002 of the auto injector 1000, thereby making it a front-loaded auto injector. In FIG. 15B, a view inside the injector 1000 showing a cassette abutting surface 1042 where against the cassette 100, 100b, 100c, 100d is resting when assembled in the injector 1000. This view shows openings in the cassette abutting surface 1042 through which internal injector parts extend during medicament delivery and the preparatory step leading to the medicament delivery. The openings include a cassette detection pin opening 1081 through which a cassette detection pin 1080 extends. The openings further include a needle safe cover pin opening 1028 through which an injector needle safe cover pin 1024 extends.

A release member opening 1043 through which a release member 1006 extends is also found in the cassette abutting surface 1042. Cassette locking tabs 1086 on the inside of the injector housing 1040 for securing the cassette 100 inside the auto injector 1000 can also be seen in FIG. 15B.

As seen most clearly in FIG. 16, the internal injector parts include a plunger rod 1018 configured for proximal movement for delivery of medicament. The plunger rod 1018 has an internal threaded surface, which is threadedly engaged with an outer threaded surface of a threaded rod 1014 positioned inside the plunger rod 1018. When the threaded rod 1014 inside the plunger rod 1018 rotates, the plunger rod 1018 is moved along the longitudinal axis L in the proximal or distal direction depending on which way the threaded rod 1014 rotates.

The auto injector 1000 comprises a release member 1006, which has two release member pins 1007 with each a track-guide protrusion 1008. The release member 1006 travels inside the index ring track 514 thereby rotating the index ring 500, when the auto injector is activated after a cassette 100, 100b, 100c, 100d is mounted in the auto injector 1000. The release member pins 1007 are connected by a release member bridge 1010 with a plunger rod opening 1012 allowing the plunger rod 1018 to pass through for medicament delivery.

The release member 1006 and the plunger rod 1018 are connected to a clutch 1030, which is configured for moving between a first clutch position where the plunger rod 1018 and the one or more release members 1006 are longitudinally coupled for proximal movement together; and a second clutch position where the plunger rod 1018 is decoupled from the one or more release members 1006 allowing the plunger rod 1018 to move longitudinally while the one or more release members 1006 are longitudinally stationary.

The auto injector 1000 further comprises an injector needle safe cover pin 1024 and a needle safe cover spring system 1026, which pushes the injector needle safe cover pin 1024 proximally. When the cassette 100, 100b, 100c, 100d is mounted in the auto injector 1000, the injector needle safe cover pin 1024 will push the second needle safe cover 440 proximally when the needle safe cover 440 is free to move proximally, and the user is not pressing the it in the distal direction simultaneously. Normally, the injector needle safe cover pin 1024 will be abutting one of the distally extending pins 454 in the needle safe cover 440.

Inside the auto injector 1000 is further a cassette detection pin 1080 and a cassette detection spring 1082, which detects when a cassette 100, 100b, 100c, 100d has been mounted in the auto injector 1000 by making contact with the other distally extending pins 454 in the needle safe cover 440.

As shown in FIG. 15A, the injector housing 1040 has a distal housing section 1044 and a cassette covering section 1046, where the distal housing section 1044 covers the internal injector parts and the proximal cassette covering section 1046 at least partly covers the cassette 100, 100b, 100c, 100d when mounted in the auto injector 1000. The distal end 104 of the cassette 100, 100b, 100c, 100d abuts a cassette abutting surface 1042 inside the auto injector 1000 when the cassette 100, 100b, 100c, 100d is mounted in the auto injector 1000.

The internal injector parts also include a drive module adapted for moving the plunger rod 1018 proximately. The drive module may be constructed in a number of manners of which the solution shown in FIG. 16 is only one example. The drive module shown in FIG. 16 includes a motor 1076 and motor gear wheels 1078 for rotating the threaded rod 1014 inside the plunger rod 1018, a battery 1056, e.g. chargeable from a USB charger connected to the printed circuit board 1054, and a number chassis parts 1070, 1077, 1090, 1094, 1095 and screws 1079 for supporting the drive module parts.

The auto injector 1000 further may include a processor controlling the drive module, the processor including the printed circuit board (PCB) 1054. Visual indicating signals to the user may be included in the form of light emitting diodes (LEDs) 1064 possibly positioned on a PCB placeholder 1062 or similar. The LEDs are displayed via an LED window frame 1050, a light box placeholder 1058, and a light guide placeholder 1060.

The auto injector may be activated by pressing an activation button 1052 positioned on a user interface housing part 1048. Normally, a protective foil 1049 is included to protect the electronic parts inside the injector 1000. For the other internal injector parts, reference is made to the reference list.

The invention is further described in the following clauses:

1. A cassette (100, 100b, 100c, 100d) for use in an auto injector for administering a medicament, wherein the cassette extends in a longitudinal direction (L) from a proximal end (102) to a distal end (104), and comprises:
a syringe holder (300, 300d) configured for receiving a syringe (200, 200d) containing the medicament, the syringe (200, 200d) comprising a seal (208) at a proximal end (202) of the syringe (200, 200d), the seal (208) being configured for being pierced by a needle (222, 222b) to obtain a fluid communication between the needle (222, 222b) and the medicament in the syringe (200, 200d);
wherein the syringe holder (300, 300d) comprises a needle assembly securing part (306) at a proximal end (302) of the syringe holder (300, 300d), wherein the needle assembly securing part (306) is configured for securing a needle assembly (220, 220b) comprising the needle (222, 222b) to the syringe holder (300, 300d) after mounting of the cassette (100, 100b, 100c, 100d) in an auto injector (1000);
a needle mounting blocking system (400, 400b), wherein a proximal end (402, 402b) of the needle mounting blocking system (400, 400b) is configured for moving between a plurality of longitudinal positions relative to the syringe holder (300, 300d), wherein the plurality of longitudinal positions at least comprises:
   a locked position (PnsL) preventing piercing of the seal (208) by the needle (222, 222b); and
   a needle assembly connecting position (PnsC) where piercing of the seal (208) by the needle (222, 222b) is allowed; and
wherein the movement of the proximal end (402, 402b) of the needle mounting blocking system (400, 400b) is configured to be controlled by the auto injector (1000) after securing the cassette (100, 100b, 100c, 100d) in the auto injector (1000).

2. The cassette according to any preceding clause, wherein the needle assembly connecting position (PnsC) is distal of the locked position (PnsL).

3. The cassette according to any preceding clause, wherein the needle assembly securing part (306) secures the needle assembly (220, 220b) to the proximal end (302) of the syringe holder (300, 300d) by a threaded connection, or a press fit connection, or a snap fit connection, or a bayonet connection, or a combination of these.

4. The cassette according to any preceding clause, wherein the needle assembly (220, 220b) is pre-mounted at the proximal end of the needle mounting blocking system (400, 400b) prior to use, and wherein the fluid connection between the syringe (200, 200d) and the needle (222, 222b) is obtained by distal movement of the needle assembly (220, 220b) and the needle mounting blocking system (400, 400b) relative to the syringe (200, 200d), possibly combined with a rotational movement of the needle assembly (220, 220b) relative to the syringe (200, 200d).

5. The cassette according to any preceding clause, wherein the cassette (100, 100b, 100c, 100d) further comprises an index ring (500, 500d) rotatable relative to the needle mounting blocking system (400, 400b) between a plurality of rotational positions including at least:
a first rotational position (R1) in which the needle mounting blocking system (400, 400b) is prevented from moving distally; and
a second rotational position (R2) in which the needle mounting blocking system (400, 400b) is moveable between the locked position (PnsL) and the needle assembly connecting position (PnsC);
   wherein rotation of the index ring (500, 500d) is controlled by movement of one or more release members (1006) in the auto injector (1000) when the cassette (100, 100b, 100c, 100d) is secured in the auto injector (1000).

6. The cassette according to clause 5, wherein the index ring (500, 500d) comprises a track (514) extending from a first corner (516) to a second corner (518) of a surface area of the index ring (500, 500d).

7. The cassette according to clause 6, wherein the track (514) extends from the first corner (516) in an angled direction towards the second corner (518), wherein the angled direction is between 20 and 70 degrees, such as between 30 and 60 degrees, such as between 35 and 55 degrees, such as between 40 and 50 degrees, such as approximately 45 degrees relative to the longitudinal direction of the cassette.

8. The cassette according to clause 7, wherein the track (514) comprises a first track section (520) and a second track section (522), wherein:
the first track section (520) extends from the first corner (516) in a first angled direction towards a first middle point (526);
the second track section (522) extends from the first middle point (526) to a second middle point (528) along the longitudinal direction of the cassette in a second angled direction;
   wherein the first angled direction is between 20 and 70 degrees, such as between 30 and 60 degrees, such as between 35 and 55 degrees, such as between 40 and 50 degrees, such as approximately 45 degrees relative to the longitudinal direction of the cassette, and
   wherein the second angled direction is between −20 and 20 degrees, such as between −10 and 10 degrees, such as approximately 0 degrees relative to the longitudinal direction of the cassette.

9. The cassette according to clause 8, wherein the track (514) further comprises a third track section (524), wherein the third track section (524) extends from the second middle point (528) in a third angled direction towards the second corner (518), wherein the third angled direction is between 20 and 70 degrees, such as between 30 and 60 degrees, such as between 35 and 55 degrees, such as between 40 and 50 degrees, such as approximately 45 degrees relative to the longitudinal direction of the cassette.

10. The cassette according to any preceding clause, wherein the plurality of longitudinal positions, which the proximal end (402, 402b) of the needle mounting blocking system (400) is moveable between, further comprises:
a medicament delivery position (PnsD) where the needle (222, 222b) is exposed;
a final locked position (PnsFL) where further longitudinal movement of the needle mounting blocking system (400, 400b) is prevented, wherein the final locked position (PnsFL) is proximal of the medicament delivery position (PnsD).

11. The cassette according to clause 10, wherein distal movement of the one or more release members (1006) after medicament delivery rotates the index ring (500, 500d) back to the first rotational position (R1), whereby the proximal end (402, 402b) of the needle mounting blocking system (400, 400b) is longitudinally locked in the final locked position (PnsFL).

12. The cassette according to any of the clauses 10-11, wherein the plurality of longitudinal positions, which the proximal end (402, 402b) of the needle mounting blocking system (400, 400b) is moveable between, further comprises:
a ready for use position (PnsR) where the proximal end (402, 402b) of the needle mounting blocking system (400, 400b) covers the needle (222, 222b), wherein the needle mounting blocking system (400, 400b) is in the ready for use position (PnsR) prior to moving to the medicament delivery position (PnsD).

13. The cassette according to any preceding clause, wherein the proximal end (402, 402b) of the needle mounting blocking system (400, 400b) is further configured as a needle safe cover covering the needle after medicament delivery.

14. The cassette according to any preceding clause, wherein the needle mounting blocking system (400, 400d) comprises:
a first needle safe cover (410, 410d);
a second needle safe cover (440, 440d), wherein the first needle safe cover (410, 410d) is displaceable relative to the second needle safe cover (440, 440d) along the longitudinal direction; and
one or more release arms (480, 580), wherein the one or more release arms (480, 580) are movable between at least two positions relative to the syringe holder (300, 300d), the at least two positions including:
   an initial position (PraIN) where the first needle safe cover (410, 410d) and the second needle safe cover (440, 440d) are separated by at the most a first separation distance; and
   a release position (PraR) where the first needle safe cover (410, 410d) and the second needle safe cover (440, 440d) are allowed to move relatively into a second separation distance being larger than the first separation.

15. The cassette according to clause 14, wherein in the initial position (PraIN), the first needle safe cover (410, 410d) is prevented from moving proximally relative to the second needle safe cover (440, 440d).

16. The cassette according to any of the clauses 14-15, wherein in the release position (PraR), the first needle safe cover (410, 410d) is allowed to move proximally relative to the second needle safe cover (440, 440d).

17. The cassette according to any of the clauses 14-16, wherein the first needle safe cover (410, 410d) comprises one or more cover locking tabs (431) locking the first needle safe cover (410, 410d) in the final locked position (PnsFL) by preventing distal movement of the first needle safe cover (410, 410d) relative to a cassette housing (600, 600d).

18. The cassette according to any of the clauses 14-17, wherein the proximal end (402, 402d) of the needle mounting blocking system (400) is comprised in the second needle safe cover (440, 440d).

19. The cassette according to clause 14-18, wherein when the proximal end (402) of the needle mounting blocking system (400) is in the locked position (PnsL) preventing piercing of the seal (208) by the needle (222), the second needle safe cover (440, 440d) is longitudinally locked relative to the syringe holder (300, 300d).

20. The cassette according to any of the clauses 14-19, wherein after securing the cassette (100, 100d) in the auto injector (1000), the second needle safe cover (440, 440d) is configured for being longitudinally unlocked, whereby the second needle safe cover (440, 440d) is moveable in the distal direction relative to the syringe holder (300, 300d) thereby moving the proximal end (402) of the needle mounting blocking system (400) into the needle assembly connecting position (PnsC) allowing for securing of the needle assembly (220) to the syringe holder (300, 300d).

21. The cassette according to any of the clauses 14-20, wherein the needle mounting blocking system (400) further comprises one or more cover springs (470) biased between the first needle safe cover (410, 410d) and the second needle safe cover (440, 440d).

22. The cassette according to clause 21, wherein after securing the cassette (100, 100d) in the auto injector (1000), the first needle safe cover (410, 410d) and the one or more cover springs (470) are configured for being longitudinally unlocked, whereby the first needle safe cover (410, 410d) and the one or more cover springs (470) are moveable in the distal direction relative to the syringe holder (300, 300d).

23. The cassette according to any of the clauses 5-22, wherein the cassette (100d) further comprises one or more index ring release tabs (330) movable relative to the index ring (500d), wherein movement of one or more release members (1006) of the auto injector into a first release member position inside the cassette (100d), when the cassette (100d) is secured in the auto injector (1000), moves the one or more index ring release tabs (330) from the initial position (PraIN) to the release position (PraR).

24. The cassette according to clause 23, wherein the one or more index ring release tabs (330) are part of the syringe holder (300d).

25. The cassette according to any of the clauses 23-24, wherein the one or more index ring release tabs (330) comprises a protruding surface (332) engaging a blocking surface (540) on the index ring (500d) in the initial position (PraIN) thereby preventing the index ring (500d) from rotating from the first rotational position (R1) to the second rotational position (R2);
wherein the one or more index ring release tabs (330) are configured for flexing radially inwardly by proximate movement of the one or more release members (1006), when the cassette (100d) is secured in the auto injector (1000);
wherein when the one or more index ring release tabs (330) flex radially inwardly, the protruding surface (332) no longer engages the blocking surface (540) on the index ring (500d) whereby the index ring (500d) is rotatable from the first rotational position (R1) to the second rotational position (R2).

26. The cassette according to clause 25 depending on any of the clauses 14-24, wherein the index ring (500d) is configured for rotating from the first rotational position (R1) to the second rotational position (R2) by movement of the first needle safe cover (410d) distally when the protruding surface (332) no longer engages the blocking surface (540) on the index ring (500d).

27. The cassette according to clause 25 or 26, wherein the index ring (500d) is configured for rotating from the first rotational position (R1) to the second rotational position (R2) by the second index ring engaging finger (469) on the second needle safe cover (440d) traveling inside a first secondary track section (550) on the index ring (500d).

28. The cassette according to any of the clauses 23-27, wherein when the one or more release members (1006) are in the first release member position inside the cassette (100d), further rotation of the index ring (500d) from the second rotational position (R2) to a third rotation position (R3) is prevented by the one or more release members (1006) engaging the first track (514) in the index ring (500d).

29. The cassette according to clause 26, wherein the index ring (500d) is rotatable from the second rotational position (R2) to the third rotation position (R3) upon proximal movement of the one or more release members (1006) of the auto injector from the first release member position inside the cassette (100d) to a second release member position inside the cassette (100d).

30. The cassette according to any of the clauses 26-29 depending on any of the clauses 14-25, wherein the index ring (500d) is configured for rotating from the second rotational position (R2) to the third second rotational position (R3) by movement of the first needle safe cover (410d) distally after proximal movement of the one or more release members (1006) of the auto injector from the first release member position inside the cassette (100d) to the second release member position inside the cassette (100d).

31. The cassette according to any of the clauses 26-30 depending on any of the clauses 14-25, wherein the index ring (500d) is configured for rotating from the second rotational position (R2) to the third second rotational position (R3) by the second index ring engaging finger (469) on the second needle safe cover (440d) traveling inside a third secondary track section (554) on the index ring (500d).

32. The cassette according to any of the clauses 26-31, wherein when the index ring (500d) is in the third second rotational position (R3), the second index ring engaging finger (469) no longer abuts the at least one of the one or more release arms (580) on the index ring (500d), whereby proximal movement of the first needle safe cover (410d) and the second needle safe cover (440d) relative to the index ring (500d) to a final locked position is allowed.

33. The cassette according to any of the clauses 14-32, wherein the release arms (580) are part of the index ring (500d).

34. The cassette according to clause 33, wherein the release arms (580) are positioned at the proximal end (502) of the index ring (500d).

35. The cassette according to any of the clauses 14-34, wherein the first needle safe cover (410d) comprises a first index ring engaging finger (433) and the second needle safe cover (440d) comprises a second index ring engaging finger (469);
wherein when the index ring (500d) is in the first rotational position (R1), the first index ring engaging finger (433) and the second index ring engaging finger (469) abut at least one of the one or more release arms (580) on the index ring (500d) preventing proximal movement of the first needle safe cover (410d) and the second needle safe cover (440d) relative to the index ring (500d); and
wherein when the index ring (500d) is in the second rotational position (R2), the first index ring engaging finger (433) no longer abuts the at least one of the one or more release arms (580) on the index ring (500d) whereby the one or more cover springs (470) are configured for moving the first needle safe cover (410d) proximately relative to the second needle safe cover (440d) thereby moving the first needle safe cover (410d) into a locked cover position where further relative longitudinal movement between the first needle safe cover (410d) and the second needle safe cover (440d) is prevented.

36. The cassette according to clause 35, wherein the first needle safe cover (410d) and the second needle safe cover (440d) comprises a first locking pair preventing proximal movement of the first needle safe cover (410d) relative to the second needle safe cover (440d) and a second locking pair preventing distal movement of the first needle safe cover (410d) relative to the second needle safe cover (440d) in the locked cover position.

37. The cassette according to any of the clauses 14-36, wherein the index ring (500d) comprises a second track (544) extending from a first secondary corner (546) to a second secondary corner (548) of a surface area of the index ring (500d), wherein the index ring (500d) is configured for rotating from the first rotational position (R1) to the second rotational position (R2) by movement of the second needle safe cover (440d) distally whereby a second index ring engaging finger (469) on the second needle safe cover (440d) travels inside the second track (544) thereby rotating the index ring (500d).

38. The cassette according to clause 37, wherein the second track (544) comprises a first secondary track section (550) and a second secondary track section (552), wherein:
the first secondary track section (550) extends from the first secondary corner (546) in a first secondary angled direction towards a first secondary middle point (526);
the second secondary track section (552) extends from the first secondary middle point (556) to a second secondary middle point (558) along the longitudinal direction of the cassette in a second secondary angle direction;
   wherein the first secondary angled direction is between 20 and 70 degrees, such as between 30 and 60 degrees, such as between 35 and 55 degrees, such as between 40 and 50 degrees, such as approximately 45 degrees relative to the longitudinal direction of the cassette, and
   wherein the second secondary angled direction is between −20 and 20 degrees, such as between −10 and 10 degrees, such as approximately 0 degrees relative to the longitudinal direction of the cassette.

39. The cassette according to clause 38, wherein the second track (544) further comprises a third secondary track section (554), wherein the third secondary track section (554) extends from the second secondary middle point (558) in a third secondary angled direction towards the second secondary corner (548), wherein the third secondary angled direction is between 20 and 70 degrees, such as between 30 and 60 degrees, such as between 35 and 55 degrees, such as between 40 and 50 degrees, such as approximately 45 degrees relative to the longitudinal direction of the cassette.

40. The cassette according to any of the clauses 1-6, wherein the index ring (500), wherein the index ring (500) is configured for rotating from the first rotational position (R1) to the second rotational position (R2) when the cassette (100) is inserted in the auto injector (1000) and the one or more release members (1006) comprised in the auto injector (1000) travels proximally inside the track (514).

41. The cassette according to any of the clauses 14-21 and 40, wherein prior to securing the cassette (100) in the auto injector (1000), the first needle safe cover (410) and the second needle safe cover (440) are allowed to move towards each other by compression of the one or more cover springs (470).

42. The cassette according to any of the clauses 14-21 and 40-41, wherein the second needle safe cover (440) comprises at least one flexible arm (446), the first needle safe cover (410) comprises a first locking tab (416), and the one or more release arms (480) each comprises a protruding surface release part (486), wherein the protruding surface release part (486) limits the proximal movement of the first needle safe cover (410) relative to the second needle safe cover (440) in the initial position (PraIN) by preventing the at least one flexible arm (446) from flexing when abutting the first locking tab (416).

43. The cassette according to any of the clauses 14-21 and 40-42, wherein upon securing the needle assembly (220) to the syringe holder (300), the second needle safe cover (440), the first needle safe cover (410) and the one or more release arms (480) are configured for moving distally relative to the syringe holder (300).

44. The cassette according to clause 43, wherein each of the second needle safe cover (440), the first needle safe cover (410) and the one or more release arms (480) moves a different distance distally relative to the syringe holder (300) upon securing the needle assembly (220) to the syringe holder (300).

45. The cassette according to any of the clauses 14-21 and 40-44, wherein the needle assembly (220) further comprises a removable needle cap (228) covering and protecting the needle (222) prior to use, wherein after securing the needle assembly (220) to the syringe holder (300) and removing the needle cap (228), the one or more cover springs (470) are adapted for pushing the first needle safe cover (410) in the proximal direction relative to the second needle safe cover (440) whereby the first needle safe cover (410) covers the needle (222) and the proximal end (402) of the needle mounting blocking system (400) is in the ready for use position (PnsR).

46. The cassette according to any of the clauses 14-21 and 40-45, wherein after securing the needle assembly (220) to the syringe holder (300), the one or more release arms (480) are locked relative to the syringe holder (300).

47. The cassette according to clause 46, wherein when the one or more release arms (480) are in the release position (PraR) after securing the needle assembly (220) to the syringe holder (300) and removing the needle cap (228), the second needle safe cover (440) is adapted for being moved proximally by the auto injector (1000) while the one or more release arms (480) are locked relative to the syringe holder (300).

48. The cassette according to any of the clauses 46-47, wherein when the one or more release arms (480) are in the release position (PraR) after securing the needle assembly (220) to the syringe holder (300) and removing the needle cap (228), the one or more cover springs (470) are adapted for pushing the first needle safe cover (410) in the proximal direction relative to the second needle safe cover (440) thereby moving the at least one flexible arm (446) proximally and past the protruding surface release part (486) into a position where the at least one flexible arm (446) flexes thereby allowing the first locking tab (416) of the first needle safe cover (410) to pass the at least one flexible arm (446) pushing the first needle safe cover (410) into an extended position relative to the second needle safe cover (440) by means of the one or more cover springs (470).

49. The cassette according to any of the clauses 14-21 and 40-48, wherein when the cassette is in the ready for use position (PnsR), the one or more locking arms (440) are in the release position (PraR) and the needle mounting blocking system (400) is in a locked cover position where further relative longitudinal movement of the first needle safe cover (410) relative to the second needle safe cover (440) is prevented.

50. The cassette according to clause 49, wherein the first needle safe cover (410) comprises a second locking tab (420) positioned distally of the first locking tab (416), and the second needle safe cover (440) comprises a stop surface (450) positioned proximally of the flexible arm (446), wherein in the locked cover position, the second locking tab (420) abuts the flexible arm (446) preventing distal movement of the first needle safe cover (410) relative to the second needle safe cover (440) and the first locking tab (416) abuts the stop surface (450) preventing proximal movement of the first needle safe cover (410) relative to the second needle safe cover (440).

51. The cassette according to clause 50, wherein the needle assembly (220) comprises a removable needle cap (228), and wherein removal of the needle cap (228) when the proximal end (402) of the needle mounting blocking system (400) is in the medicament delivery position (PnsD) will allow the proximal end (402) of the needle mounting blocking system (400) to move proximally.

52. The cassette according to any of the clauses 1-8, wherein the plurality of longitudinal positions, which the proximal end (402b) of the needle mounting blocking system (400b) is moveable between, further comprises:
a medicament delivery position (PnsD) where the needle (222) is exposed;
a final locked position (PnsFL) where further longitudinal movement of the needle mounting blocking system (400b) is prevented,
   wherein the medicament delivery position (PnsD) and the final locked position (PnsFL) are at the same longitudinal position.

53. The cassette according to clause 52, wherein after medicament delivery, distal movement of the one or more release members (1006) in the auto injector (1000) is configured for rotating the index ring (500) back to the first rotational position (R1), whereby the proximal end (402b) of the needle mounting blocking system (400b) is longitudinally locked in the final locked position (PnsFL).

54. A system for use in an auto injector for administering a medicament, the system comprising:
a cassette according to any of the clauses 1-53;
a syringe (200, 200d) containing the medicament and a seal (208) at a proximal end (202) of the syringe (200, 200d), the seal (208) being configured for being pierced by a distal end of a needle (222) to obtain a fluid communication between the medicament in the syringe (200, 200d) and the needle (222, 222b); and
a needle assembly (220, 220b) comprising the needle (222, 222b), a needle assembly adaptor (226, 226b) for securing the needle assembly (220, 220b) to the syringe holder (300, 300d), and a removable needle cap (228) covering and protecting the needle (222);
   wherein prior to securing the cassette (100, 100d) in the auto injector (1000), the needle mounting blocking system (400, 400b) prevents the distal end of the needle (222, 222b) from piercing the seal (208).

55. The system according to clause 54, wherein the needle assembly (220b) further comprises a needle assembly shield (235b) configured for being in a plurality of positions including:
an initial delivery position, where the needle assembly shield (235b) of the needle assembly (220b) covers the needle (222b);
a medicament delivery position, where the needle (222b) is exposed;
a final locked position, where the needle assembly shield (235b) of the needle assembly (220b) covers the needle (222b).

56. The system according to any of the clauses 54-55 further comprising the auto injector (1000), wherein the auto injector (1000) is configured for receiving the cassette (100, 100d).

57. The system according to clause 56, wherein the auto injector (1000) comprises one or more release members (1006) configured for moving into the cassette (100, 100d) when the cassette has been received in the auto injector (1000).

58. An auto injector (1000) for administering a medicament, the auto injector (1000) being configured for receiving any of the cassettes (100, 100d) according to the clauses 1-53 and/or any of the systems according to clauses 54-55 without requiring modifications to the auto injector (1000).

59. A system comprising an auto injector (1000) for administering a medicament, the auto injector (1000) being configured for receiving any of the cassettes (100, 100d) according to the clauses 1-53 without requiring modifications.

60. The system of clause 59 further comprising a needle assembly (220, 220b) comprising the needle (222, 222b), a needle assembly adaptor (226) for securing the needle assembly (220, 220b) to the syringe holder (300, 300d), and a removable needle cap (228) covering and protecting the needle (222, 222b), wherein prior to securing the cassette (100, 100d) in the auto injector (1000), the needle mounting blocking system (400, 400b) prevents a distal end of the needle (222, 222b) from piercing the seal (208).

61. The system according to clause 60, wherein the needle assembly (220) further comprises a needle assembly shield (235b) configured for being in a plurality of positions including:
an initial delivery position, where the needle assembly shield (235b) of the needle assembly (220b) covers the needle (222b);
a medicament delivery position, where the needle (222b) is exposed;
a final locked position, where the needle assembly shield (235b) of the needle assembly (220b) covers the needle (222b).

### REFERENCES

100, 100b, 100c, 100d cassette
102, 102b, 102c proximal end of the cassette
104, 104b, 104c distal end of the cassette
200, 200d syringe
202 proximal end of the syringe
204 distal end of the syringe
206, 206d syringe compartment
208 seal/septum at the syringe outlet end
209 syringe outlet
212 first stopper
214 first sub-compartment
216 second sub-compartment
217 by-pass region
218 second stopper
220, 220b needle assembly
222, 222b needle
224, 224b needle hub
226, 226b needle assembly adaptor
228, 228b needle cap/rigid needle shield/RNS
232, 232b proximal end of the needle
234, 234b distal end of the needle
235b needle assembly shield of the needle assembly
236 rigid needle shield, RNS
300, 300d syringe holder
302 proximal end of the syringe holder
304 distal end of the syringe holder
306 needle assembly locking part
308 elongated tubular part
310 index ring receiving opening
312 window allowing a view of the syringe
314 release arm receiving channel
315 release arm blocking surface
316 distal ring part
318 plunger opening
319 release member opening
320 cassette detection tab
322 syringe support surface
324 syringe securing hook
330 index ring release arm on the syringe holder
332 protruding surface
400, 400b needle safe cover assembly/needle mounting blocking system
402, 402b proximal end of the needle safe cover assembly/needle mounting blocking system
404b distal end of the needle safe cover assembly/needle mounting blocking system
408 combined fully extended needle safe cover
410, 410d first needle safe cover
412 proximal end of the first needle safe cover
414 distal end of the first needle safe cover
416 first locking tab on the first needle safe cover
417 proximal sloping surface of the first locking tab
418 distal surface of the first locking tab
420 second locking tab on the first needle safe cover
421 proximal sloping surface of the second locking tab
422 distal surface of the second locking tab
424 proximally positioned surface plate
426 opening at the proximal end of the first needle safe cover
428 inner support surface
429 cassette housing guide tab
430 cover locking tab
431 distal end of the cover locking tab
432 distally extending pin
433 first index ring engaging finger
434 first needle safe cover arm
435 first needle safe cover interface
436 distal first needle safe cover locking surface
437 proximal first needle safe cover locking surface
438 cassette housing stop surface
439 sloping surface on the first needle safe cover arm
440, 440′, 440b, 440d second needle safe cover
442 proximal end of the second needle safe cover
444 distal end of the second needle safe cover
446 flexible arm on the second needle safe cover
447 distal end of the flexible arm
448 sloping surface on the flexible arm
449 proximal stop surface on the flexible arm
450 stop surface on the second needle safe cover
452, 452b tubular part
454 proximally extending pin
456, 456b distally extending pin
457,457b distal end of the distally extending pin
458 inspection opening
460,460b first locking recess
461b second locking recess
462, 462b safe cover locking protrusion/blocking system locking protrusion
464, 464b surface of the distally extending pin
465 proximal end surface
466 proximal second needle safe cover locking tab
467 distal second needle safe cover locking tab
468 second needle safe cover interface
469 second index ring engaging finger
470 cover spring
472 proximal end of the cover spring
474 distal end of the cover spring
480 release arm
482 proximal end of the release arm
484 distal end of the release arm
486 protruding surface release part on the release arm
490 flexible fingers
500, 500d index ring
502 proximal end of the index ring
504 distal end of the index ring
506 index ring locking protrusion
508 index ring arm
510 tubular part of the index ring
512 surface area of the index ring
514 track in the surface area of the index ring
516 first corner of the track
518 second corner of the track
520 first section of the track
522 second section of the track
524 third section of the track
526 first middle point of the track
528 second middle point of the track
540 blocking surface
544 second track
546 first secondary corner
548 second secondary corner
550 first secondary track of the second track
552 second secondary track of the second track
554 third secondary track of the second track
555 fourth secondary track of the second track
556 first secondary middle point of the second track
558 second secondary middle point of the second track
580 release arm on the index ring
600,600d cassette housing
602 proximal end of the cassette housing
604 distal end of the cassette housing
606 housing locking protrusion
608 sloping surface of the housing locking protrusion
610 proximal surface of the housing locking protrusion
612 opening in the cassette housing
614 inspection window
616 cassette securing tabs
618d first needle safe cover stop surface
620d first needle safe cover channel
1000 auto injector
1002 proximal end of the auto injector
1004 distal end of the auto injector
1006 release member
1007 release member pin
1008 track-guide protrusion
1010 release member bridge
1012 plunger rod opening
1014 threaded rod
1016 motor and threaded rod connector
1018 plunger rod
1019 stopping clutch tab
1020 first plunger rod plug part
1022 second plunger rod plug part engaging the stopper
1024 injector needle safe cover pin
1026 needle safe cover spring system
1028 needle safe cover pin opening in the cassette abutting surface
1030 clutch
1032 switch
1040 injector housing
1042 cassette abutting surface
1043 release member opening in the cassette abutting surface
1044 distal housing section
1046 cassette covering section
1048 first user interface housing part
1049 protective foil
1050 LED window frame
1052 activation button
1054 Print circuit board (PCB)
1055 RCT battery
1056 battery
1057 adhesive
1058 light box placeholder
1060 light guide placeholder
1062 PCB placeholder
1064 LED placeholder with an LED
1066 content management system (CMS)
1068 USB gasket
1070 proximal chassis part
1076 motor
1077 motor flange
1078 motor gear
1079 screw
1080 cassette detection pin
1081 cassette detection pin opening in the cassette abutting surface
1082 cassette detection spring
1084 cassette detect holder
1086 cassette locking tab in the injector housing
1088 NFC antenna for detection of the RFID chip on the cassette
1090 distal end part connecting to the housing
1092 bearing
1094 middle chassis part
1095 distal chassis part
L longitudinal direction of the cassette and auto injector
Pns12L position where the first needle safe cover and the second needle safe cover are locked
Pns1IN unlocked initial position
PnsL locked position preventing piercing of the seal by the needle
PnsC needle assembly connecting position
PnsR ready for use position
PnsD medicament delivery position
PnsFL final locked position
PraIN initial position of the release arm
PraR release position of the release arm
R1 first rotational position of the index ring
R2 second rotational position of the index ring
R3 third rotational position of the index ring

1. A cassette (100, 100d) for use in an auto injector for administering a medicament, wherein the cassette extends in a longitudinal direction (L) from a proximal end (102) to a distal end (104), and comprises:
a syringe holder (300, 300d) configured for receiving a syringe (200, 200d) containing the medicament, the syringe (200, 200d) comprising a seal (208) at a proximal end (202) of the syringe (200, 200d), the seal (208) being configured for being pierced by a needle (222) to obtain a fluid communication between the medicament in the syringe (200, 200d) and the needle (222);
wherein the syringe holder (300, 300d) comprises a needle assembly securing part (306) at a proximal end (302) of the syringe holder (300, 300d), wherein the needle assembly securing part (306) is configured for securing a needle assembly (220) comprising the needle (222) to the syringe holder (300, 300d) after mounting of the cassette (100, 100d) in an auto injector (1000);
a first needle safe cover (410, 410d);
a second needle safe cover (440, 440d), wherein the first needle safe cover (410, 410d) is displaceable relative to the second needle safe cover (440, 440d) along the longitudinal direction (L); and
one or more release arms (480, 580), wherein the one or more release arms (480, 580) are movable between at least two positions relative to the syringe holder (300, 300d), the at least two positions including:
an initial position (PraIN) where the first needle safe cover (410, 410d) and the second needle safe cover (440, 440d) are separated by at the most a first separation distance; and
a release position (PraR) where the first needle safe cover (410, 410d) and the second needle safe cover (440, 440d) are allowed to move relatively into a second separation distance being larger than the first separation.

2. The cassette according to claim 1, wherein in the initial position (PraIN), the first needle safe cover (410, 410d) is prevented from moving proximally relative to the second needle safe cover (440, 440d).

3. The cassette according to claim 1, wherein in the release position (PraR), the first needle safe cover (410, 410d) is allowed to move proximally relative to the second needle safe cover (440, 440d).

4. The cassette according to claim 1, wherein the cassette (100, 100d) further comprises one or more cover springs (470) biased between the first needle safe cover (410, 410d) and the second needle safe cover (440, 440d).

5. (canceled)

6. The cassette according to claim 1, wherein the first needle safe cover (410, 410d) is movable between a plurality of longitudinal positions relative to the syringe holder (300, 300d), the plurality of longitudinal positions including:
a ready for use position (PnsR);
a medicament delivery position (PnsD) where the needle (222) is exposed, wherein the medicament delivery position (PnsD) is distally of the ready for use position (PnsR);
a final locked position (PnsFL) where further longitudinal movement of the first needle safe cover (410) is prevented, wherein the final locked position (PnsFL) is proximal of the medicament delivery position (PnsD).

7-8. (canceled)

9. The cassette according to claim 1, wherein the second needle safe cover (440, 440d) further functions as a needle mounting blocking system (400) configured for moving between:
a locked position (PnsL) preventing piercing of the seal (208) by the needle (222) prior to securing the cassette (100, 100d) in the auto injector (1000); and
a needle assembly connecting position (PnsC) where piercing of the seal (208) by the needle (222) is allowed after securing the cassette (100, 100d) in the auto injector (1000);
wherein the movement of the second needle safe cover (440, 440d) is configured to be controlled by the auto injector (1000) after securing the cassette (100, 100d) in the auto injector (1000).

10-12. (canceled)

13. The cassette according to claim 1, wherein the needle assembly securing part (306) secures the needle assembly (220) to the proximal end (302) of the syringe holder (300, 300d) by a threaded connection, or a press fit connection, or a snap fit connection, or a bayonet connection, or a combination of these.

14. The cassette according to claim 1, wherein the cassette (100, 100d) further comprises an index ring (500, 500d) rotatable relative to first needle safe cover (410, 410d) between a plurality of rotational positions including at least:
a first rotational position (R1) in which the second needle safe cover (440, 440d) is prevented from moving distally; and
a second rotational position (R2) in which the second needle safe cover (440, 440d) is longitudinally moveable;
wherein movement of one or more release members (1006) in the auto injector when the cassette (100, 100d) is secured in the auto injector (1000) allows for rotation of the index ring (500, 500d).

15. The cassette according to claim 14, wherein the index ring (500, 500d) comprises a first track (514) extending from a first corner (516) to a second corner (518) of a surface area of the index ring (500, 500d).

16-20. (canceled)

21. The cassette according to claim 1, wherein the needle assembly (220) is pre-mounted at the first needle safe cover (410, 410d) prior to use, and wherein the fluid connection between the syringe (200, 200d) and the needle (222) is obtained by distal movement of the needle assembly (220), the second needle safe cover (440, 440d) and the first needle safe cover (410, 410d) relative to the syringe (200, 200d), possibly combined with a rotational movement of the needle assembly (220) relative to the syringe (200, 200d).

22-30. (canceled)

31. The cassette according to claim 14, wherein the cassette further comprises one or more index ring release tabs (330) movable relative to the index ring (500d), wherein movement of one or more release members (1006) of the auto injector into a first release member position inside the cassette (100d), when the cassette (100d) is secured in the auto injector (1000), moves the one or more index ring release tabs (330d) from the initial position (PraIN) to the release position (PraR).

32-53. (canceled)

54. System for use in an auto injector for administering a medicament, the system comprising:
a cassette (100, 100d) according to claim 1; and
a syringe (200, 200d) comprising a syringe compartment (206, 206d) containing the medicament and a seal (208) at a proximal end (202) of the syringe (200, 200d), the seal (208) being configured for being pierced by a distal end of a needle (222) to obtain a fluid communication between the medicament in the syringe (200, 200d) and the needle (222);
wherein prior to securing the cassette (100, 100d) in the auto injector (1000), the first needle safe cover (410, 100d) prevents the distal end of the needle (222) from piercing the seal (208).

55-60. (canceled)

61. A cassette (100, 100d) for use in an auto injector for administering a medicament, wherein the cassette extends in a longitudinal direction (L) from a proximal end (102) to a distal end (104), and comprises:
a needle safe cover (400, 410, 410d, 440, 440d);
an index ring (500, 500d) rotatable relative to the needle safe cover (400, 410, 410d, 440, 440d) between a plurality of rotational positions including at least:
a first rotational position (R1) in which the needle safe cover (400, 410, 410d, 440, 440d) is prevented from moving distally; and
a second rotational position (R2) in which the needle safe cover (400, 410, 410d, 440, 440d) is longitudinally moveable;
a syringe holder (300, 300d) configured for receiving a syringe (200, 200d) containing the medicament, the syringe (200, 200d) comprising a seal (208) at a proximal end (202) of the syringe (200, 200d), the seal (208) being configured for being pierced by a needle (222) to obtain a fluid communication between the medicament in the syringe (200, 200d) and the needle (222);
wherein the syringe holder (300, 300d) comprises a needle assembly securing part (306) at a proximal end (302) of the syringe holder (300, 300d), wherein the needle assembly securing part (306) is configured for securing a needle assembly (220) comprising the needle (222) to the syringe holder (300, 300d) after mounting of the cassette (100, 100d) in an auto injector (1000);
one or more index ring release tabs (330, 330d) movable between at least two positions relative to the index ring (500, 500d), the at least two positions including:
an initial position (PraIN) where the index ring (500, 500d) is prevented from rotating from the first rotational position (R1) to the second rotational position (R2); and
a release position (PraR) where the index ring (500, 500d) is rotatable from the first rotational position (R1) to the second rotational position (R2);
wherein movement of one or more release members (1006) of the auto injector into a first release member position inside the cassette (100, 100d), when the cassette (100, 100d) is secured in the auto injector (1000), moves the one or more index ring release tabs (330) from the initial position (PraIN) to the release position (PraR).

62. The cassette according to claim 61, wherein when the one or more release members (1006) are in the first release member position inside the cassette (100, 100d), further rotation of the index ring (500, 500d) from the second rotational position (R2) to a third rotation position (R3) is prevented by the one or more release members (1006) engaging a first track (514) in the index ring (500, 500d).

63. The cassette according to claim 61, wherein the one or more index ring release tabs (330) are part of the syringe holder (300, 300d).

64. The cassette according to claim 61, wherein the one or more index ring release tabs (330) comprises a protruding surface (332) engaging a blocking surface (540) on the index ring (500, 500d) in the initial position (PraIN) thereby preventing the index ring (500, 500d) from rotating from the first rotational position (R1) to the second rotational position (R2);
wherein the one or more index ring release tabs (330) are configured for flexing radially inwardly by proximate movement of the one or more release members (1006), when the cassette (100, 100d) is secured in the auto injector (1000);
wherein when the one or more index ring release tabs (330) flex radially inwardly, the protruding surface (332) no longer engages the blocking surface (540) on the index ring (500, 500d) whereby the index ring (500, 500d) is rotatable from the first rotational position (R1) to the second rotational position (R2).

65. The cassette according to claim 61, wherein the index ring (500, 500d) is rotatable from the first rotational position (R1) to the second rotational position (R2) by movement of the needle safe cover (400, 410, 410d, 440, 440d) distally.

66. The cassette according to claim 61, wherein the index ring (500, 500d) comprises a second track (544) extending from a first secondary corner (546) to a second secondary corner (548) of a surface area of the index ring (500, 500d), wherein the index ring (500, 500d) is configured for rotating from the first rotational position (R1) to the second rotational position (R2) by movement of the needle safe cover (400, 410, 410d, 440, 440d) distally whereby at least one protruding part on the needle safe cover (400, 410, 410d, 440, 440d) travels inside the second track (544) thereby rotating the index ring (500, 500d).

67. The cassette according to claim 61, wherein the needle safe cover (400, 410, 410d, 440, 440d) is configured for moving between:
a locked position (PnsL) preventing piercing of the seal (208) by the needle (222) prior to securing the cassette (100, 100d) in the auto injector (1000); and
a needle assembly connecting position (PnsC) where piercing of the seal (208) by the needle (222) is allowed after securing the cassette (100, 100d) in the auto injector (1000);
wherein when the index ring (500, 500d) is in the first rotational position (R1), the needle safe cover (400, 410, 410d, 440, 440d) is in to the locked position (PnsL) and
wherein when the index ring (500, 500d) is in the second rotational position (R2), the needle safe cover (400, 410, 410d, 440, 440d) is movable the needle assembly connecting position (PnsC) by movement of the needle safe cover (400, 410, 410d, 440, 440d) distally.

68. The cassette according to claim 61, wherein the needle safe cover (400, 410, 410d, 440, 440d) comprises:
a needle safe cover (410, 410d); and
a second needle safe cover (440, 440d), wherein the first needle safe cover (410, 410d) is displaceable relative to the second needle safe cover (440, 440d) along the longitudinal direction,
wherein in the initial position (PraIN), the first needle safe cover (410, 410d) and the second needle safe cover (440, 440d) are separated by at the most a first separation distance, and wherein in the release position (PraR), the first needle safe cover (410, 410d) and the second needle safe cover (440, 440d) are allowed to move relatively into a second separation distance being larger than the first separation.

69-78. (canceled)
